(19) United States
(12) Patent Application Publication
(10) Pub. No.: US 2015/0025412 A1
(43) Pub. Date: Jan. 22, 2015

(54) ALLERGY TESTING SYSTEM, METHOD AND KIT

(71) Applicants: Sherwin A. Gillman, Santa Ana, CA (US); Alfredo Jalowayski, West Palm Beach, FL (US)

(72) Inventors: Sherwin A. Gillman, Santa Ana, CA (US); Alfredo Jalowayski, West Palm Beach, FL (US)

(21) Appl. No.: 14/338,106

(22) Filed: Jul. 22, 2014

Related U.S. Application Data
(60) Provisional application No. 61/857,108, filed on Jul. 22, 2013.
(60) Provisional application No. 62/002,094, filed on May 22, 2014.

(51) Int. Cl. A61B 5/00

(52) U.S. Cl. CPC A61B 5/411
USPC 600/556

(57) ABSTRACT

An allergy skin test kit includes a template having puncture site indicators and a computer-readable storage medium storing allergy test information that associates the puncture site indicators of the template with template locations or test substance indicators. A method of performing an allergy skin test includes providing a template having puncture site indicators. Allergy test information that associates the puncture site indicators of the template with template locations or test substances identifiers is obtained. Image data corresponding to a test area on the skin of a test subject obtained and image regions corresponding to the puncture site indicators of the template are identified. Test results corresponding to the image regions are determined based, at least in part, on the allergy test information.

### BACKGROUND

1. Technical Field

The present invention generally relates to allergy testing, and more particularly to computer-based assessment of allergy skin test results.

2. Description of the Related Art

Skin testing can be used to diagnose many common types of allergies. During a typical skin test, extracts of test substances that can produce allergic reactions are placed just beneath the skin of a test subject. After a predetermined amount of time has elapsed, a medical professional evaluates areas near where the test substances were introduced into the skin of the test subject. If the test subject has an allergic reaction to a particular test substance, a red, raised lump or wheal surrounded by an inflamed area or flare (e.g., erythema) may appear on the skin in an area near where the test substance was placed. The medical professional can measure the size of the wheal and the size of the flare and use those measurements to determine a test result indicating the degree to which the test subject is allergic to the test substance. For example, the medical professional may assign a score from 0 to 4 to indicate the degree to which the test subject is allergic to the test substance, wherein a score of 0 indicates no reaction and a score of 4 indicates an extremely severe reaction. After the medical professional has evaluated each area near where one of the test substances was introduced to the skin of the test subject, the medical professional may generate a written medical report documenting the results of the allergy skin test.

Conventional allergy skin testing can be time consuming. Before an allergy skin test is administered, a medical professional typically prepares a tray having wells for holding extracts of test substances for which the allergy skin test is being conducted. A small quantity of one of the test substances is placed into one of the wells of the tray. Applicators are then prepared, wherein each applicator includes at least one sharp tip for puncturing the skin of the test subject and introducing one of the test substances. The tip of each applicator is dipped into one of the wells and is covered with one of the test substances. The medical professional then punctures a location on the skin of the test subject with one of the applicators. This process is repeated until each of the test substances has been applied to the skin of the test subject via respective applicators. After a predetermined amount of time (e.g., 15 minutes) has elapsed the medical professional evaluates areas of the skin of the test subject near where the test substances have been introduced. In each area where a reaction is observed, the medical professional may assign a score or measure the size of the resulting wheal and the size of the resulting flare for the test substance that caused the reaction.

### BRIEF SUMMARY

The results of allergy skin testing are subjective and can vary among medical professionals. For example, different medical professionals may assign different scores to the same test area. Additionally, the sizes of the wheal and the flare are critical to assigning a test score for a test substance. Subjectivity or even a mistake made in assessing the size of the wheal and/or the size of the flare, may result in a test score that does not provide an accurate indication of the degree to which the test subject is allergic to a particular test substance.

There is therefore a need for tools that enable skin testing to be performed faster and more accurately. Additionally, there is a need for tools that apply evaluation criteria consistently when determining results of allergy skin tests.

An allergy testing kit may be summarized as including: a template including a plurality of puncture site indicators arranged at a plurality of predetermined template locations; and a non-transitory computer-readable storage medium storing allergy test information that associates the plurality of puncture site indicators of the template with the plurality of predetermined template locations or a plurality of test substance identifiers.

The allergy test information may associate the plurality of puncture site indicators of the template with the plurality of predetermined template locations. The allergy test information may associate the plurality of puncture site indicators of the template with the plurality of test substance identifiers. The template may include an allergy test type indicator and the allergy test information may include allergy test type information corresponding to the allergy test type indicator. The allergy test type indicator may be in a text format. The allergy test type indicator may be a barcode. The allergy test type indicator may be a radio-frequency identification (RFID) tag. Each of the puncture site indicators may include at least one aperture formed in a surface of the template. Each of the puncture site indicators may include at least one pre-inked marking for transferring to skin of a test subject. Each of the puncture site indicators may include at least one projection for transferring ink to skin of a test subject. The allergy testing kit may further include: a tray including a plurality of wells for holding a plurality of test substances. The tray may include a first allergy test type indicator indicating a first allergy test type identifier and the template may include a second allergy test type indicator indicating a second allergy test type identifier, and the first allergy test type identifier may be the same as the second allergy test type identifier. The tray may include a plurality of test substance indicators corresponding to the plurality of wells. The template may include a plurality of test substance indicators corresponding to the plurality of test substance indicators of the tray. The allergy test information may indicate that at least one of the puncture site indicators is for at least one control test substance. The allergy test information may indicate that a first puncture site indicator is for a negative control test substance and a second puncture site indicator is for a positive control test substance. The allergy testing kit may further include: a reference marker having a predetermined shape and a predetermined size. The template may include a reference marker having a predetermined shape and a predetermined size. The reference marker may include at least one aperture formed in the template. The reference marker may include at least one projection for applying ink to the skin of a test subject. The reference marker may include at least one pre-inked marking for transferring to the skin of a test subject.

A method of performing a skin test may be summarized as including: providing a template that includes a plurality of puncture site indicators; obtaining allergy test information that associates the plurality of puncture site indicators of the template with a plurality of template locations or a plurality of test substance identifiers; obtaining image data corresponding to one or more images of a test area on the skin of a test subject; identifying a plurality of image regions of the image data corresponding to the plurality of puncture site indicators of the template; and determining a plurality of test results corresponding to the plurality of puncture site indicators of the template based, at least in part, on the allergy test information.

The allergy test information may associate the plurality of puncture site indicators of the template with the plurality of template locations. The allergy test information may associate the plurality of puncture site indicators of the template with the plurality of test substance identifiers. The method may further include: obtaining an allergy test type identifier or a template type identifier, wherein the plurality of image regions of the image data corresponding to the plurality of puncture site indicators is identified, at least in part, using the allergy test type identifier or the template type identifier. The allergy test type identifier or the template type identifier may be obtained via a camera unit. The allergy test type identifier or the template type identifier may be obtained via a barcode reader. The allergy test type identifier or the template type identifier may be obtained via a radio frequency identification (RFID) reader. The determining of the plurality of test results may include determining at least one dimension of at least one wheal and at least one dimension of at least one flare. The image data may include a plurality of puncture site marking regions and the plurality of image regions of the image data corresponding to the plurality of puncture site indicators may be identified, at least in part, on the plurality of puncture site marking regions. The allergy test information may be associated with an identifier of the template. The method may further include: providing a tray including a plurality of wells corresponding to the plurality of puncture site indicators of the template. The method of claim 22 may further include: providing a computer-readable storage medium storing the allergy test information.

A method of performing an allergy skin test may be summarized as including: providing a template including a plurality of puncture site indicators; obtaining layout information of the plurality of puncture site indicators of the template; obtaining an allergy test type identifier or a template type identifier; and storing allergy test information associating the allergy test type identifier or the template type identifier and the layout information in a computer-readable storage medium.

The allergy test type identifier may be obtained; and the allergy test information may associate the allergy test type identifier with the layout information. The template type identifier may be obtained; and the allergy test information may associate the template type identifier with the layout information. The method may further include: obtaining a plurality of test substance identifiers corresponding to the plurality of puncture site indicators, wherein the allergy test information associates the plurality of test substance identifiers with the plurality of puncture site indicators. The layout information may include location information for each of the plurality of puncture site indicators of the template. The method may further include: obtaining image data; and identifying a plurality image regions of the image data corresponding to the plurality of puncture site indicators of the template, at least in part, using the allergy test information. The method may further include: determining a plurality of test results corresponding to the plurality of image regions. The method may further include: associating the plurality of test results with a plurality of test substance identifiers.

An allergy testing system may be summarized as including: at least one processor; at least one non-transitory processor-readable storage medium that stores processor-executable instructions that, when executed, cause the at least one processor to: obtain first image data that corresponds to an image which includes a wheal region and a flare region; obtain a first plurality of coordinates that respectively correspond to a first plurality of points disposed about a periphery of the wheal region, the first plurality of coordinates which define a plurality of vertices of a first polygon; obtain a second plurality of coordinates that respectively correspond to a second plurality of points disposed about a periphery of the flare region, the second plurality of coordinates which define a plurality of vertices of a second polygon; obtain a first value based at least in part on the first plurality of coordinates; and obtain a second value based at least in part on the second plurality of coordinates.

The at least one storage medium may store instructions that, when executed, further cause the at least one processor to: cause at least the first value and the second value to be stored in one or more processor-readable storage media. The at least one storage medium may store instructions that, when executed, further cause the at least one processor to: obtain a third plurality of coordinates that respectively correspond to a third plurality of points disposed about the periphery of the wheal region, wherein to obtain the first plurality of coordinates the at least one processor performs a first ant colony optimization on at least some of the third plurality of coordinates; and obtain a fourth plurality of coordinates that respectively correspond to a fourth plurality of points disposed about the periphery of the flare region, wherein to obtain the second plurality of coordinates the at least one processor performs a second ant colony optimization on at least some of the fourth plurality of coordinates. To obtain the first value the at least one processor may at least in part determine a first plurality of dimensions of the first polygon, and to obtain the second value the at least one processor may at least in part determine a second plurality of dimensions of the second polygon. To obtain the first value the at least one processor may at least in part determine a sum of the first plurality of dimensions of the first polygon, and to obtain the second value the at least one processor may at least in part determine a sum of the second plurality of dimensions of the second polygon. To obtain the first value the at least one processor may at least in part determine a length of a first line segment that corresponds to a first dimension of the first polygon and a length of a second line segment that corresponds to a second dimension of the first polygon, the second line segment orthogonal to the first line segment, and to obtain the second value the at least one processor may at least in part determine a length of a third line segment that corresponds to a first dimension of the second polygon and a length of a fourth line segment that corresponds to a second dimension of the second polygon, the fourth line segment orthogonal to the third line segment. The first dimension of the first polygon may be a longest dimension of the first polygon and the second line segment may pass through a midpoint of the first line segment, and the first dimension of the second polygon may be a longest dimension of the second polygon and the fourth line segment may pass through a midpoint of the third line segment. The first plurality of coordinates may be arranged in a first sequence such that when sequential pairs of the first plurality of coordinates are interconnected with a first plurality of line segments the first polygon is formed, and the second plurality of coordinates may be arranged in a second sequence such that when sequential pairs of the second plurality of coordinates are interconnected with a second plurality of line segments the second polygon is formed. The at least one storage medium may store instructions that, when executed, further cause the at least one processor to: generate second image data that corresponds to an image that includes the wheal region, the flare region, the first polygon, and the second polygon. The at least one storage medium may store instructions that, when executed, further cause the at least one processor to: cause the second image data to be displayed. The at least one storage medium may store instructions that, when executed, further cause the at least one processor to: obtain an indication of a first color; and obtain an indication of a second color, wherein the second image data includes an indication that the first polygon has the first color and an indication that the second polygon has the second color. The at least one storage medium may store instructions that, when executed, further cause the at least one processor to: generate second image data that corresponds to an image that includes the wheal region, the flare region, the first polygon, the second polygon, the first value, and the second value. The at least one storage medium may store instructions that, when executed, further cause the at least one processor to: obtain an indication of a region of interest, wherein first plurality of coordinates and the second plurality of coordinates are obtained based on at least part of the first image data which corresponds to at least part of the image within the region of interest. The at least one storage medium may store instructions that, when executed, further cause the at least one processor to: receive test information; and cause the first value, the second value, and at least some of the test information to be stored in one or more processor-readable storage media. The test information may include at least one of: an indication of a type of allergen, an indication of a puncture or a prick type of allergic reaction test, an indication of an intradermal type of allergic reaction test, and an indication of coordinates that correspond to a region of an image. The at least one storage medium may store instructions that, when executed, further cause the at least one processor to: perform at least one operation on the first image data that smooths the first image data. The at least one storage medium may store instructions that, when executed, further cause the at least one processor to: perform at least one operation on the first image data that reduces noise in the image that corresponds to the first image data. The at least one storage medium may store instructions that, when executed, further cause the at least one processor to: obtain a third value based at least in part on the first value and the second value.

### BRIEF DESCRIPTION OF THE SEVERAL VIEWS OF THE DRAWINGS

FIG. 1 is a schematic diagram of an example computer system of an allergy testing system according to one illustrated embodiment.

FIG. 2 is a diagram of a template of an allergy testing system according to one illustrated embodiment.

FIGS. 3A and 3B are front and rear views, respectively, of a template shown in FIG. 2.

FIG. 4 is a diagram of tray of an allergy testing system according to one illustrated embodiment.

FIGS. 5A and 5B are respective side views of the tray shown in FIG. 4.

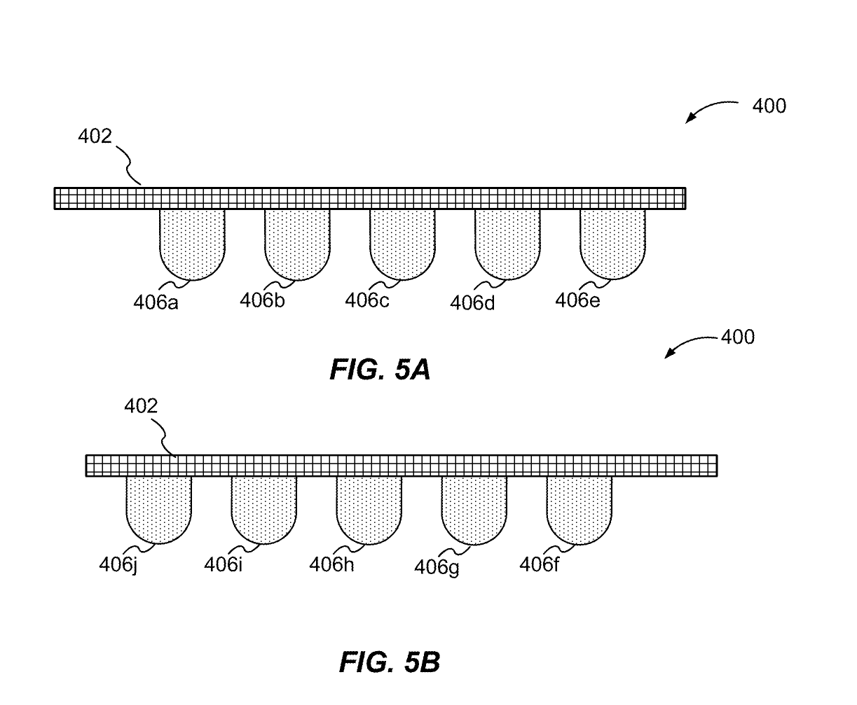

FIG. 6 illustrates a method of generating allergy test information according to one illustrated embodiment.

FIG. 7 is a method for performing an allergy skin test according to one illustrated embodiment.

FIG. 8 is a diagram of a template of an allergy testing system according to one illustrated embodiment.

FIG. 9 is a diagram of a test area of the skin of a test subject after the template shown in FIG. 8 has been used to apply puncture site markers according to one illustrated embodiment.

FIG. 10 is a diagram of the test area of the skin of the test subject shown in FIG. 9 after test substances have been applied according to one illustrated embodiment.

FIG. 11 is a diagram of the test area of the skin of the test subject shown in FIG. 10 after several allergic reactions have occurred according to one illustrated embodiment.

FIG. 12 is a diagram of an image of the test area of the skin of the test subject shown in FIG. 11 according to one illustrated embodiment.

FIG. 13 illustrates a method of generating test results according to one illustrated embodiment.

FIG. 14 is a diagram of a test area of the skin of a test subject after several allergic reactions have occurred according to one illustrated embodiment.

FIG. 15 is a diagram of an image cropped based on a region of interest according to one illustrated embodiment.

FIG. 16 is diagram of an interface that an operator may use to specify testing information according to one illustrated embodiment.

FIG. 17 is diagram of an allergic reaction with first points disposed about the periphery of a wheal region and second points disposed about the periphery of a flare region according to one illustrated embodiment.

FIG. 18 is a diagram of a first polygon and a second polygon according to one illustrated embodiment.

FIG. 19 is a diagram of a test result according to one illustrated embodiment.

FIG. 20 is a diagram of a spreadsheet in which test results may be stored according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with computing systems including client and server computing systems, as well as networks, including various types of telecommunications networks, have not been shown or described in detail to avoid unnecessarily obscuring descriptions of the embodiments.

Unless the context requires otherwise, throughout the specification and claims which follow, the word “comprise” and variations thereof, such as “comprises” and “comprising,” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

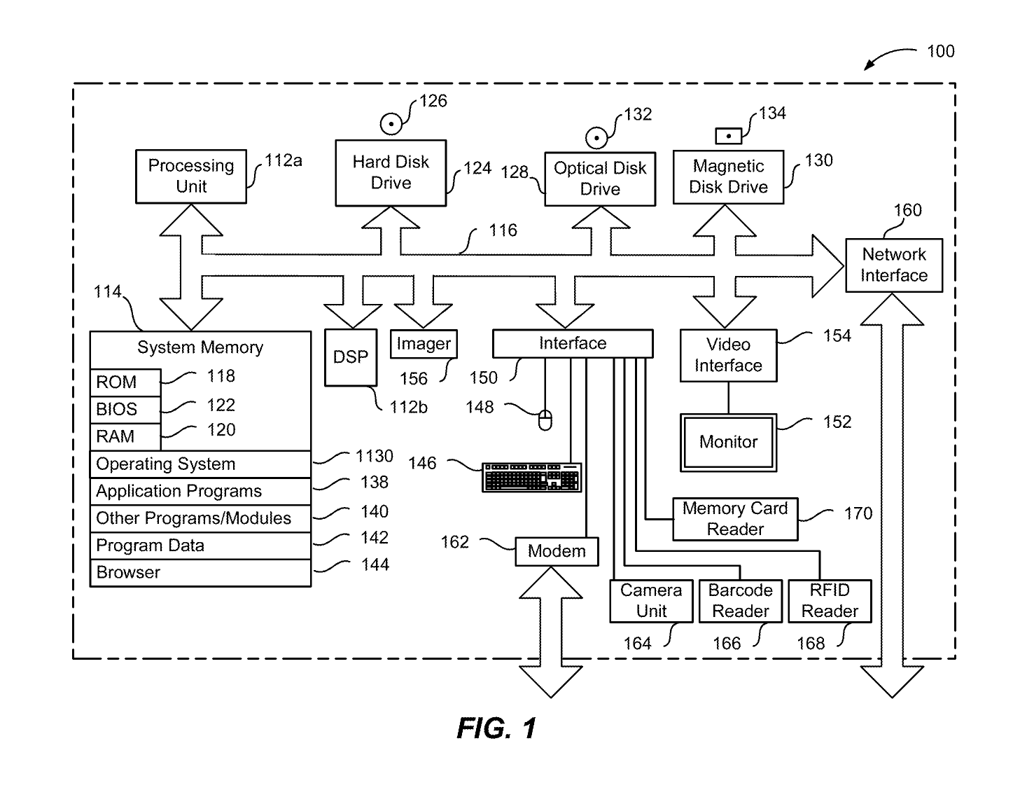

FIG. 1 shows an example computer system 100, according to one illustrated embodiment. The computer system 100 is suitable for implementing systems, devices and methods for performing allergy tests, according to one illustrated embodiment. The computer system 100 will at times be referred to in the singular herein, but this is not intended to limit the embodiments to a single device, since in typical embodiments, there may be more than one computer system or device involved. Unless described otherwise, the construction and operation of the various blocks shown in FIG. 1 are of conventional design. As a result, such blocks need not be described in further detail herein, as they will be understood by those skilled in the relevant art.

The computer system 100 may include one or more processing units 112a, 112b (collectively 112), a system memory 114 and a system bus 116 that couples various system components including the system memory 114 to the processing units 112. The processing units 112 may be any logic processing unit, such as one or more central processing units (CPUs) 112a, digital signal processors (DSPs) 112b, application-specific integrated circuits (ASICs), field programmable gate arrays (FPGAs), etc. The system bus 116 can employ any known bus structures or architectures, including a memory bus with memory controller, a peripheral bus, and a local bus. The system memory 114 includes read-only memory (ROM) 118 and random access memory (RAM) 120. A basic input/output system (BIOS) 122, which can form part of the ROM 118, contains basic routines that help transfer information between elements within the computer system 100, such as during start-up.

The computer system 100 may also include a plurality of interfaces such as network interface 160 and interface 150 supporting modem 162 or any other wireless/wired interfaces.

The computer system 100 may include a hard disk drive 124 for reading from and writing to a hard disk 126, an optical disk drive 128 for reading from and writing to removable optical disks 132, and/or a magnetic disk drive 130 for reading from and writing to magnetic disks 134. The optical disk 132 can be a CD-ROM, while the magnetic disk 134 can be a magnetic floppy disk or diskette. The hard disk drive 124, optical disk drive 128 and magnetic disk drive 130 may communicate with the processing unit 112 via the system bus 116. The hard disk drive 124, optical disk drive 128 and magnetic disk drive 130 may include interfaces or controllers (not shown) coupled between such drives and the system bus 116, as is known by those skilled in the relevant art. The drives 124, 128 and 130, and their associated computer-readable storage media 126, 132, 134, may provide non-volatile and non-transitory storage of computer-readable instructions, data structures, program modules and other data for the computer system 100. Although the depicted computer system 100 is illustrated employing a hard disk drive 124, optical disk drive 128 and magnetic disk drive 130, those skilled in the relevant art will appreciate that other types of computer-readable storage media that can store data accessible by a computer may be employed, such as magnetic cassettes, flash memory, Bernoulli cartridges, RAMs, ROMs, smart cards, etc. For example, computer-readable storage media may include, but is not limited to, random access memory (RAM), read-only memory (ROM), electrically erasable programmable read-only memory (EEPROM), flash memory, compact disc ROM (CD-ROM), digital versatile disks (DVD) or other optical disk storage, magnetic cassettes, magnetic tape, magnetic disk storage or other magnetic storage devices, solid state memory or any other medium which can be used to store the desired information and which may be accessed by processing unit 112a.

Program modules can be stored in the system memory 114, such as an operating system 136, one or more application programs 138, other programs or modules 140 and program data 142. Application programs 138 may include instructions that cause the processor(s) 112 to generate allergy test information, to analyze skin test data, and to prepare associated reports. Other program modules 140 may include instructions for handling security such as password or other access protection and communications encryption. The system memory 114 may also include communications programs, for example, a Web client or browser 144 for permitting the computer system 100 to access and exchange data with sources such as Web sites of the Internet, corporate intranets, extranets, or other networks and devices as described herein, as well as other server applications on server computing systems. The browser 144 in the depicted embodiment is markup language based, such as Hypertext Markup Language (HTML), Extensible Markup Language (XML) or Wireless Markup Language (WML), and operates with markup languages that use syntactically delimited characters added to the data of a document to represent the structure of the document. A number of Web clients or browsers are commercially available such as those from Mozilla, Google, and Microsoft.

While shown in FIG. 1 as being stored in the system memory 114, the operating system 136, application programs 138, other programs/modules 140, program data 142 and browser 144 can be stored on the hard disk 126 of the hard disk drive 124, the optical disk 132 of the optical disk drive 128 and/or the magnetic disk 134 of the magnetic disk drive 130.

An operator can enter commands and information into the computer system 100 through input devices such as a touch screen or keyboard 146 and/or a pointing device such as a mouse 148, and/or via a graphical user interface. Other input devices can include a microphone, joystick, game pad, tablet, scanner, etc. These and other input devices are connected to one or more of the processing units 112 through an interface 150 such as a serial port interface that couples to the system bus 116, although other interfaces such as a parallel port, a game port or a wireless interface or a universal serial bus (USB) can be used. The interface 150 also couples a camera unit 164, a barcode reader 166, a radio frequency identification (RFID) tag reader 168, and a memory card reader 170 to the system bus 116. A monitor 152 or other display device is coupled to the system bus 116 via a video interface 154, such as a video adapter. An image capture device or imager 156 is coupled to the system bus 116. The image 156 can scan a physical image and provide corresponding image data to the processing unit 112a. The computer system 100 can include other devices, such as speakers, printers, etc.

The computer system 100 can operate in a networked environment using logical connections to one or more remote computers and/or devices as described above with reference to FIG. 1. For example, the computer system 100 can operate in a networked environment using logical connections to one or more mobile devices, landline telephones and other service providers or information servers. Communications may be via a wired and/or wireless network architecture, for instance wired and wireless enterprise-wide computer networks, intranets, extranets, telecommunications networks, cellular networks, paging networks, and other mobile networks.

It should be understood that the various techniques described herein may be implemented in connection with hardware, software and/or firmware or, where appropriate, with a combination of such. Thus, the methods and apparatuses of the disclosure, or certain aspects or portions thereof, may take the form of program code (i.e., instructions) embodied in tangible media, such as floppy diskettes, CD-ROMs, DVDs, hard drives, flash drives, or any other machine-readable or processor-readable storage medium wherein, when the program code is loaded into and executed by a machine, such as a processor of a computer or mobile device, the machine becomes an apparatus for practicing various embodiments. In the case of program code execution on programmable computers or mobile devices, such generally includes a processor, a storage medium readable by the processor (including volatile and non-volatile memory and/or storage elements), at least one input device, and at least one output device. One or more programs may implement or utilize the processes described in connection with the disclosure, e.g., through the use of an API, reusable controls, or the like. Such programs are preferably implemented in a high level procedural or object-oriented programming language to communicate with a computer system including the computer system 100 depicted in FIG. 1. However, the program(s) can be implemented in assembly or machine language, if desired. In any case, the language may be a compiled or interpreted language, and combined with hardware implementations.

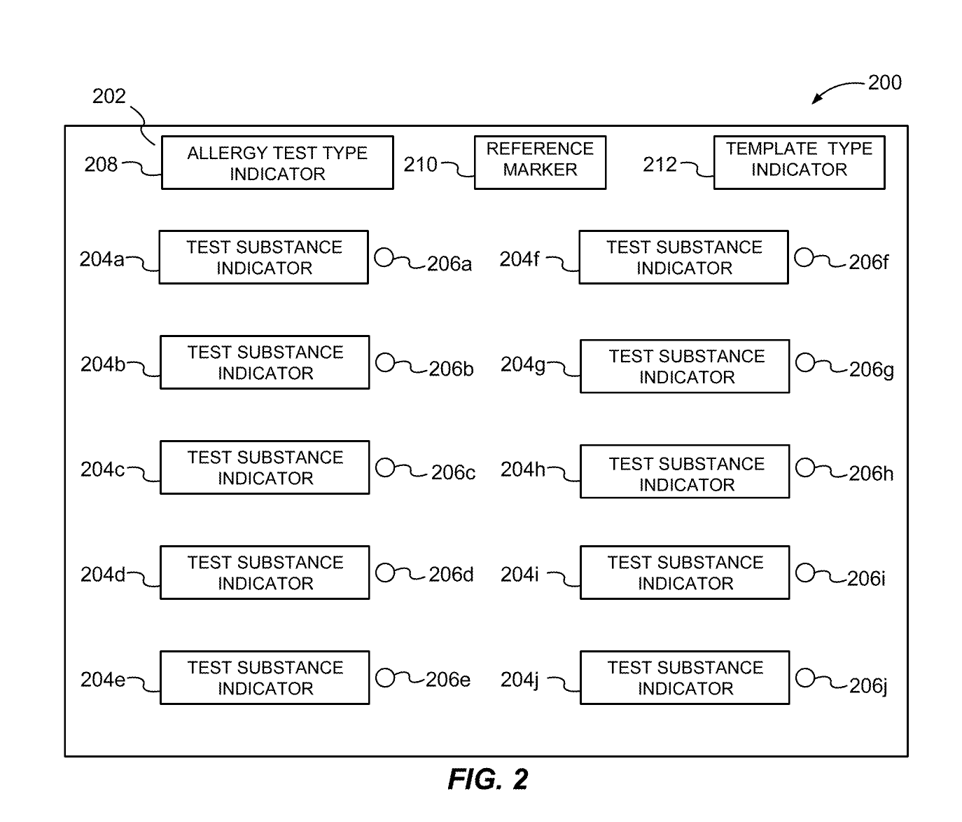

FIG. 2 is a diagram of a template 200 according to one illustrated embodiment. The template 200 includes a panel 202, a plurality of test substance indicators 204a-204j, a plurality of corresponding puncture site indicators 206a-206j, an allergy test type indicator 208, a reference marker 210, and a template type indicator 212. In one embodiment, the panel 202 of the template 200 is formed from a thin (e.g., 2 millimeters) plastic material. In one embodiment, the panel 202 is formed from cardboard and has a thin layer of a coating material (e.g., plastic) formed over surfaces thereof. In one embodiment, the panel 202 is formed from paper. In one embodiment, the panel 202 is formed from a fabric material (e.g., polyester).

Each of the test substance indicators 204a-204j identifies a test substance to be applied to the skin of a test subject at a puncture site near a marking formed using one of the puncture site indicators 206a-206j. For illustrative simplicity, the template 200 includes only ten test substance indicators 204a-204j and corresponding puncture site indicators 206a-206j. The template 200 may include virtually any number of test substance indicators and corresponding puncture site indicators without departing from the scope of the present disclosure.

The test substance indicators 204a-204j can take the form of numbers, letters, symbols, patterns, and/or shapes that identify the test substances to be applied to the skin of a test subject in a test area. In one embodiment, the test substance indicators 204a-204j include index numbers that identify an order in which the test substances are to be applied to the skin of the test subject. In one embodiment, the test substance indicators 204a-204j include letters that identify the names of the test substances that are to be applied to the skin of the test subject. In one embodiment, the test substance indicators 204a-204j include index numbers and letters that identify the names of the test substances that are to be applied to the skin of a test subject.

In one embodiment, the test substance indicators 204a-204j are stencils (i.e., openings or apertures formed through the panel 202) that enable a medical professional to transfer the names of substances (or corresponding index numbers, patterns, and/or symbols) to the skin of the test subject using a pen, a marker, or a paint that is sprayed on or applied with a brush. In one embodiment, the test substance indicators 204a-204j are projections extending from the bottom surface of the template 200 in the shapes of letters, numbers, and/or symbols to which an ink can be applied and transferred to the skin of a test subject when the test substance indicators 204a-204j contact the skin. In one embodiment, the test substance indicators 204a-204j are pre-inked markings that include letters, numbers, and/or symbols formed on the bottom surface of the panel 202 that can be transferred to the skin of the test subject. For example, after a protective layer is removed from the bottom surface of the panel 202, the bottom surface of the panel 202 is placed in contact with the skin of a test subject and a slight force is applied to the upper surface of the panel 202 to transfer the markings onto the skin of the test subject.

The puncture site indicators 206a-206j can take the form of shapes that are used to create puncture site markings that identify areas on the skin of the test subject where the test substances are to be applied with applicators. In one embodiment, the puncture site indicators 206a-206j are circular stencils that enable a medical professional to transfer circular markings to the skin of a test subject using a pen, a marker, or a paint that is sprayed on or applied with a brush. In one embodiment, the puncture site indicators 206a-206j are circular projections extending from the bottom surface of the panel 202 to which an ink can be applied and transferred to the skin of the test subject when the puncture site indicators 206a-206j contact the skin of the test subject. In one embodiment, the puncture site indicators 206a-206j are pre-inked circular markings formed on the bottom surface of the panel 202 that can be transferred to the skin of a test subject. For example, after a protective layer is removed from the bottom surface of the panel 202, the bottom surface of the panel 202 is placed on the skin of a test subject and a slight force is applied to the upper surface of the panel 202 to transfer the markings onto the skin of the test subject.

The allergy test type indicator 208 identifies a particular type of allergy test. For example, the test type indicator 208 may indicate at least one of the following types of allergy tests: a dust allergy test, a food allergy test, a fungi allergy test, an insect allergy test, a mite allergy test, and a pollen allergy test. In one embodiment, the allergy test type indicator 208 is a label attached to or printed on the panel 202. In one embodiment, the test type indicator 208 is a barcode or other machine-readable indicator attached to or printed on the panel 202. In one embodiment, the test type indicator 208 is an RFID tag attached to or embedded within the panel 202. In one embodiment, the test type indicator 208 is detachable from the template and can be placed on the skin of a test subject in a test area. For example, the test type indicator 208 is a sticker that can be removed from the panel 202 and attached to the skin of a test subject in a test area. In one embodiment, the allergy test type indicator 208 includes one or more stencils that enable a medical professional to transfer markings to the skin of a test subject in a test area using a pen, a marker, or a paint that is sprayed on or applied with a brush. In one embodiment, the allergy test type indicator 208 includes projections extending from the bottom surface of the panel 202 to which an ink can be applied and transferred to the skin of a test subject when the allergy test type indicator 208 contacts the skin of the test subject. In one embodiment, the test type indicator 208 includes pre-inked markings formed on the bottom surface of the panel 202 that can be transferred to the skin of a test subject. For example, after a protective layer is removed from the bottom surface of the panel 202 and the template 200 is placed on the skin of the test subject to transfer the markings onto the skin of the test subject.

The reference marker 210 may be of a predetermined size, location, and/or orientation with respect to the panel 202. For example, the reference marker 210 is transferred to the skin of a test subject and used to identify a reference point and/or a reference size on the skin of the test subject. In one embodiment, the reference marker 210 is a label attached to or printed on the panel 202. In one embodiment, the reference marker 210 is detachable from the template and can be placed on the skin of a test subject in a test area. For example, the reference marker 210 is a fiducial marker in the form of a sticker having a predetermined size, shape, and color that can be removed from the panel 202 and attached to the skin of a test subject. In one embodiment, the reference marker 210 includes one or more stencils that enable a medical professional to transfer markings (e.g., crosshairs) to the skin of a test subject using a pen, a marker, or a paint that is sprayed on or applied with a brush. In one embodiment, the reference marker 210 includes pre-inked markings formed on the bottom surface of the panel 202 that can be transferred to the skin of a test subject. For example, after a protective layer is removed from the bottom surface of the panel 202, the bottom surface of the panel 202 is placed on the skin of a test subject and a slight force is applied to the top surface of the panel 202 to transfer the markings onto the skin of the test subject.

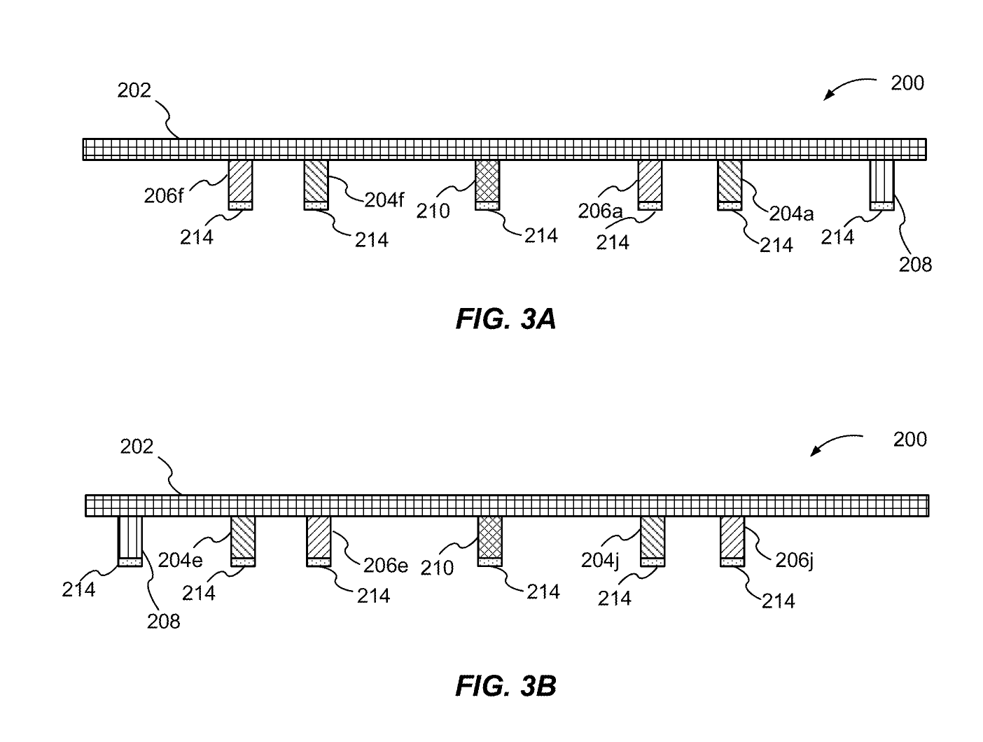

In one embodiment, the reference marker 210 includes one or more projections extending from the bottom surface of the panel 202 to which an ink can be applied and transferred to the skin of a test subject when the reference marker 210 contacts the skin of the test subject. For example, FIGS. 3A and 3B illustrate an embodiment in which the test substance indicators 204a-204j, the puncture site indicators 206a-206j, the allergy test type indicator 208, and the reference marker 210 of the template 200 are projections. Only the test substance indicators 204a, 204e, 204f, and 204j and the puncture site indicators 206a, 206e, 206f, and 206j, can be seen in FIGS. 3A and 3B. An ink 214 is applied to the bottom surfaces of the projections. When the projections are placed in contact with the skin of a test subject, the ink 214 is transferred to the skin of the test subject forming markings on the skin. In one embodiment, the projections include pre-inked markings. That is, the template 200 is provided with the ink 214 on the projections.

The template type indicator 212 identifies a particular type of the template 200. The template type indicator 212 may be encoded in a data carrier, for example in a machine-readable symbol (e.g., linear barcode symbol, 2-D barcode symbol), radio frequency identification (RFID) wireless transponder, magnetic stripe, or the like. The data carrier may be carried on or in the panel 202.

In one embodiment, the template type indicator 212 is a label attached to or printed on the panel 202. In one embodiment, the template type indicator 212 is a barcode or other machine-readable code attached to or printed on the panel 202. In one embodiment, the template type indicator 212 is an RFID tag attached to or embedded within the panel 202. In one embodiment, the template type indicator 212 is detachable from the template and can be placed on the skin of a test subject in a test area. For example, the template type indicator 212 is a sticker that can be removed from the panel 202 and attached to the skin of a test subject in a test area. In one embodiment, the template type indicator 212 includes one or more stencils that enable a medical professional to transfer markings to the skin of a test subject using a pen, a marker, or a paint that is sprayed on or applied with a brush. In one embodiment, the template type indicator 212 includes projections extending from the bottom surface of the panel 202 to which an ink can be applied and transferred to the skin of the test subject when the template type indicator 212 contacts the skin of the test subject. In one embodiment, the template type indicator 212 includes pre-inked markings formed on the bottom surface of the panel 202 that can be transferred to the skin of a test subject. For example, after a protective layer is removed from the bottom surface of the panel 202, the bottom surface of the panel 202 is placed on the skin of a test subject and a slight force is applied to the top of the panel 202 to transfer the markings to the skin of the test subject.

In one embodiment, the template type indicator 212 indicates a particular layout and/or geometry of the puncture site indicators 206a-206j of the template 200. For example, the template type indicator 212 has a format of C-R-S, where C identifies a number of columns of test site indicators, R identifies a number of rows of test site indicators, and S identifies a size of the template 200 (e.g., small, medium, or large). The computer system 100 may store layout information for each of a plurality of types of templates 200 in the program data 142 of the system memory 114.

In one illustrated embodiment, the computer system 100 stores layout information for the template 200 including an index number and a location (e.g., coordinates in units of millimeters) of each of the puncture site indicators 206a-206j. For example, the puncture site indicators 206a-206j may be circles and the location for each puncture site indicator includes coordinates of the center of a corresponding circle. In one embodiment, a predetermined location of the reference marker 210 is used as the origin of the coordinate system for the coordinates of the puncture site indicators 206a-206j. For example, the reference marker 210 includes crosshairs and the origin of the coordinate system corresponds to a location or point where the crosshairs intersect. Additionally, the crosshairs can include one or more arrows that point in one or more predetermined directions. In one embodiment, the computer system 100 stores layout information that includes the information shown in Table 1.

TABLE 1
Template Type ID = 2-5-M

Column

| Row | 1 | 2 |
|---|---|---|
| 1 | Index Number = 1 | Index Number = 6 |
| | Location = (−50, 30) | Location = (50, 30) |
| 2 | Index Number = 2 | Index Number = 7 |
| | Location = (−50, 60) | Location = (50, 60) |
| 3 | Index Number = 3 | Index Number = 8 |
| | Location = (−50, 90) | Location = (50, 90) |
| 4 | Index Number = 4 | Index Number = 9 |
| | Location = (−50, 120) | Location = (50, 120) |
| 5 | Index Number = 5 | Index Number = 10 |
| | Location = (−50, 150) | Location = (50, 150)) |

The layout information can include additional information. For example, each of the puncture site indicators 206a-206j may have a different size, shape, color, and/or pattern associated therewith and the layout information includes information identifying the size, shape, color, and/or pattern of each of the puncture site indicators 206a-206j.

In one embodiment, the computer system 100 stores allergy test information that includes layout information for the template 200 and test substance information corresponding to the puncture site indicators 206a-206j. For example, the computer system 100 stores allergy test information indicating, for each of the puncture site indicators 206a-206j, an index number, a test substance identifier, type information, and a location (e.g., coordinates in units of millimeters). For example, the type information indicates whether the test substance is a potential allergen, a negative control substance, or a positive control substance. In one embodiment, the computer system 100 stores allergy test information that includes the information shown in Table 2.

TABLE 2
Test Type ID = 1478932
Test Name = Food
Template Type ID = 2-5-M

Column

| Row | 1 | 2 |
|---|---|---|
| 1 | Index Number = 1 | Index Number = 6 |
| | Identifier = Saline | Identifier = Milk |
| | Type = Negative | Type = Potential |
| | Control Location = | Allergen Location = |
| | (−50, 30) | (50, 30) |
| 2 | Index Number = 2 | Index Number = 7 |
| | Identifier = Apple | Identifier = Mushroom |
| | Type = Potential | Type = Potential |
| | Allergen Location = | Allergen Location = |
| | (−50, 60) | (50, 60) |
| 3 | Index Number = 3 | Index Number = 8 |
| | Identifier = Bean | Identifier = Potato |
| | Type = Potential | Type = Potential |
| | Allergen Location = | Allergen Location = |
| | (−50, 90) | (50, 90) |
| 4 | Index Number = 4 | Index Number = 9 |
| | Identifier = Corn | Identifier = Rice |
| | Type = Potential | Type = Potential |
| | Allergen Location = | Allergen Location = |
| | (−50, 120) | (50, 120) |
| 5 | Index Number = 5 | Index Number = 10 |
| | Identifier = Garlic | Identifier = Histamine |
| | Type = Potential | Type = Positive |
| | Allergen Location = | Control Location = |
| | (−50, 150) | (50, 150) |

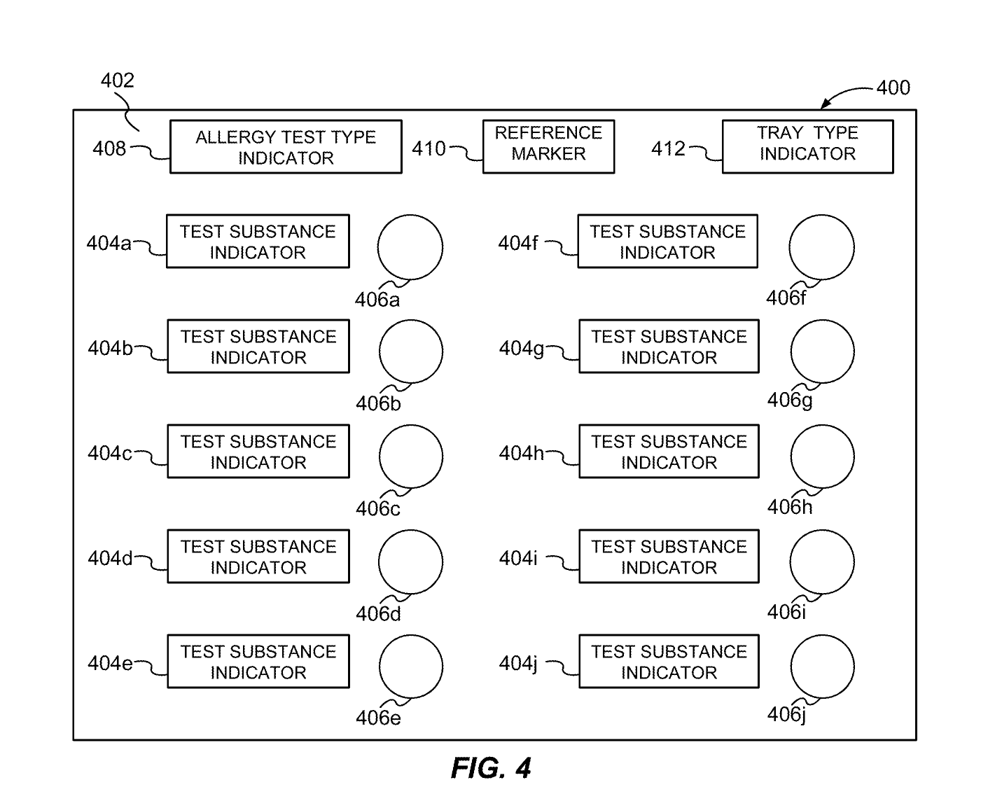

FIG. 4 is a diagram of a tray 400 according to one illustrated embodiment. The tray 400 includes a panel 402, a plurality of test substance indicators 404a-404j, a plurality of corresponding wells 406a-406j, an allergy test type indicator 408, a reference marker 410, and a tray type indicator 412. In one embodiment, the test substance indicators 404a-404j of the tray 400 are the same as the test substance indicators 204a-204j of the template 200. The wells 406a-406j include portions that extend from the bottom surface of the panel 402, as shown in FIGS. 4A and 4B. Prior to performing an allergy test, a medical professional puts predetermined amounts of test substances corresponding to the test substance indicators 404a-404j into the wells 406a-406j. For example, if the test substance indicator 404b indicates soy, a predetermined amount of a soy extract is put into the well 406b. After the test substances have been put into the wells 406a-406j, applicators are placed into the wells 406a-406j such that at least one puncture tip of one of the applicators is covered by the test substance included in one of the wells 406a-406j.

The reference marker 410 of the tray 400 indicates a relative orientation of the tray 400. For example, the reference marker 410 indicates the top of the tray 400. The tray type indicator 412 identifies a type of the tray 400. In one embodiment, the tray type indicator identifies a number of columns of the wells 406a-406j, a number of rows, and/or a size (e.g., small, medium, or large) of the tray 400. For example, tray type indicator 412 is a label having “2-5-M” printed thereon to indicate that the tray 400 has 2 columns, 5 rows, and is of a medium size.

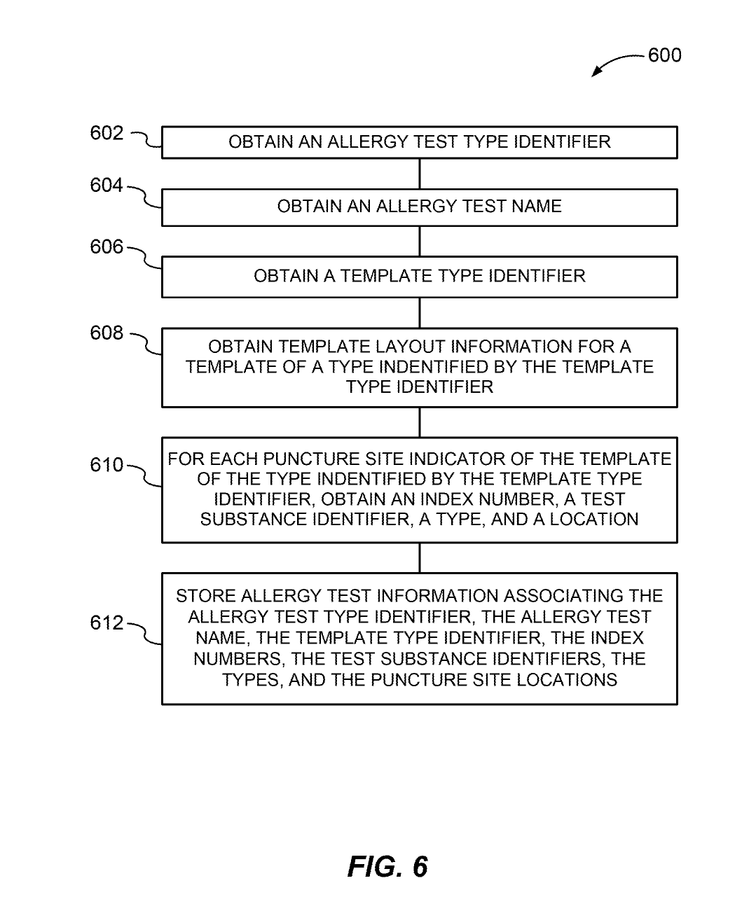

FIG. 6 illustrates a flowchart of a process 600 for generating allergy test information, according to one non-limiting embodiment. The process 600 may be performed by the computer system 100 shown in FIG. 1. For example, an allergy test information setup program stored in the application programs 138 of the system memory 114 is copied onto the RAM 120 and is executed by the processing unit 112a to generate the allergy test information.

At 602, an allergy test type identifier is obtained. In one embodiment, the processing unit 112a executing the allergy test setup program uses a predefined rule to generate a system unique allergy test identifier. For example, the system unique allergy test identifier has an integer value and the processing unit 112a generates a new allergy test type identifier by incrementing the most recently generated allergy test type identifier by one. In one embodiment, the processing unit 112a obtains the allergy test type identifier from the keyboard 146. For example, an operator of the computer system 100 enters the test type identifier via the keyboard 146 in response to a prompt that is displayed by the monitor 152.

At 604, an allergy test name is obtained. In one embodiment, the processing unit 112a receives the allergy test type name from the keyboard 146. For example, an operator of the computer system 100 enters the allergy test type name using the keyboard 146 in response to a prompt that is displayed by the monitor 152.

At 606, a template type identifier is obtained. In one embodiment, the processing unit 112a receives the template type identifier from the keyboard 146. For example, an operator of the computer system 100 enters the template type identifier using the keyboard 146 in response to a prompt that is displayed by the monitor 152. In one embodiment, the processing unit 112a obtains the template type identifier from the barcode reader 166. For example, the template type indicator 212 of the template 200 may be in the form of a barcode, and an operator of the computer system 100 scans the template type indicator 212 of the template 200 using the barcode reader 166, which transmits the corresponding template type identifier to the processing unit 112a. In one embodiment, the processing unit 112a obtains the template type identifier from the RFID reader 168. For example, the template type indicator 212 of the template 200 is in the form of an RFID tag, and an operator of the computer system 100 scans the template type indicator 212 of the template 200 using the RFID reader 168, which transmits the corresponding template type identifier to the processing unit 112a.

At 608, template layout information is obtained. In one embodiment, the processing unit 112a obtains the template layout information from the program data 142 of the system memory 114. For example, the processing unit 112a obtains information corresponding to Table 1 that is associated with the template type identifier obtained at 606, from the program data 142 of the system memory 114. In one embodiment, the processing unit 112a obtains the template layout information from the keyboard 146. For example, an operator of the computer system 100 enters the template layout information using the keyboard 146 in response to a series of prompts that are displayed by the monitor 152.

At 610, an index number, a test substance identifier, a test substance type, and a location are obtained for each puncture site indicator of a template of the type obtained at 606. In one embodiment, the processing unit 112a obtains the test substance identifiers and test substance types from the keyboard 146 and obtains the index numbers and locations from template layout information stored in the program data 142 of the system memory 114. In one embodiment, the processing unit 112a obtains the index numbers, test substance identifiers, test substance types, and puncture site indicator locations from the keyboard 146. For example, an operator of the computer system 100 enters the index numbers, test substance identifiers, test substance types, and puncture site indicator locations using the keyboard 146 in response to a series of prompts that are displayed by the monitor 152.

At 612, allergy test information is stored, wherein the allergy test information associates the test type identifier obtained at 602, the allergy test name obtained at 604, the template type identifier obtained at 606, the layout information obtained at 608, and the index numbers, test substance identifiers, test substance types, and the site indicator locations obtained at 610. For example, the processing unit 112a causes the information included in Table 2 to be stored in the program data 142 of the system memory 114 at 612.

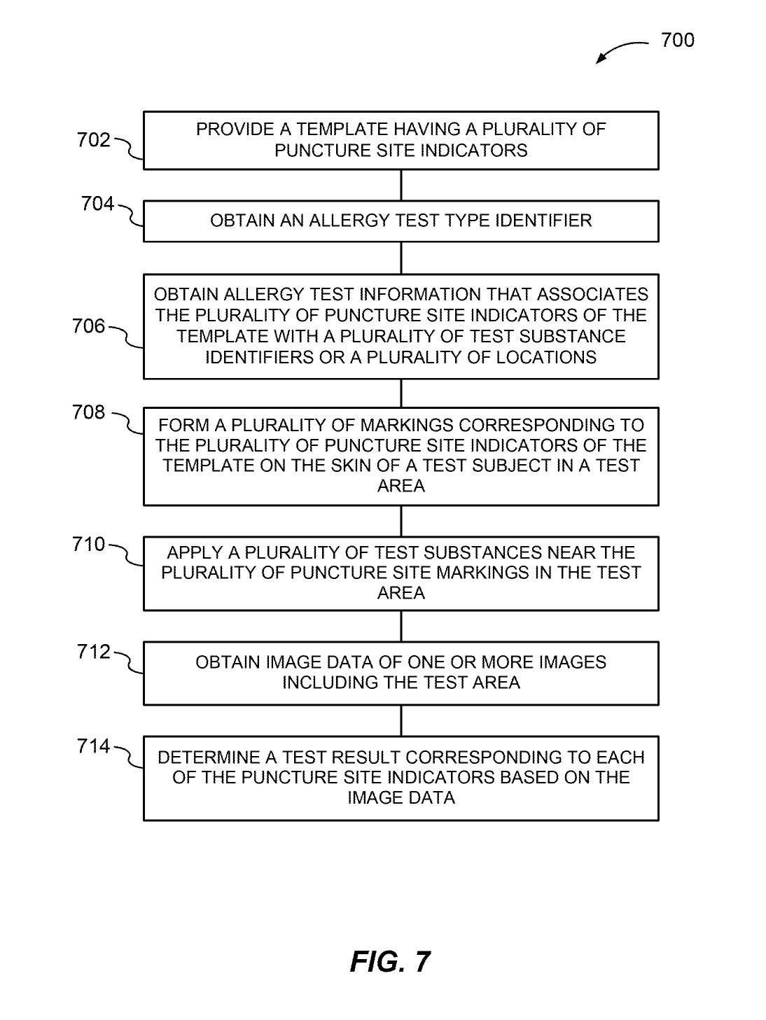

A process for performing an allergy test according one illustrated embodiment will be described now with reference to FIGS. 7-11. FIG. 7 illustrates a flowchart of a process 700 for performing an allergy test, according to one non-limiting embodiment. One or more acts of the process 700 may be performed by the computer system 100 shown in FIG. 1. For example, an allergy test results program stored in the application programs 138 of the system memory 114 is copied onto the RAM 120 and is executed by the processing unit 112a to perform one or more acts of the process 700.

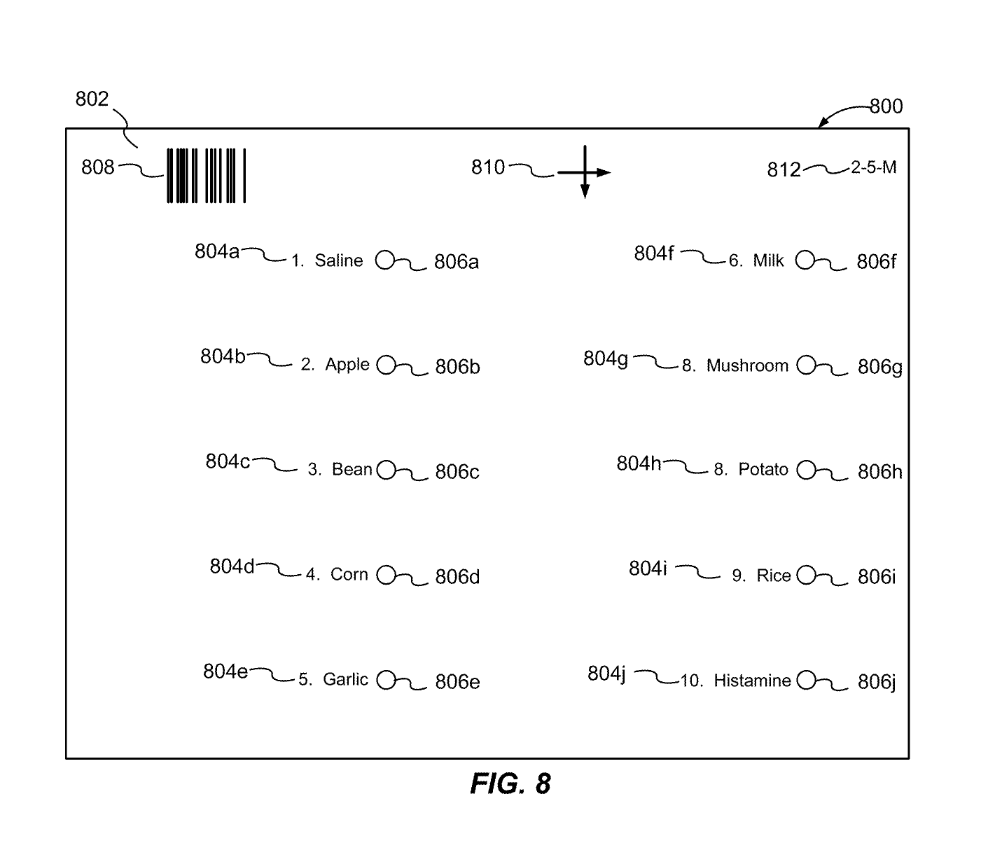

At 702, a template having a plurality of puncture site indicators is provided. For example, FIG. 8 illustrates a template 800 that is provided at 702. The template 800 includes a panel 802, test substance indicators 804a-804j, corresponding puncture site indicators 806a-806j, an allergy test type indicator 808, a reference marker 810, and a template type indicator 812. Each of the test substance indicators 804a-804j includes text representing an index number and a name of a test substance. Each of the puncture site indicators 806a-806j is a circular stencil or aperture through which a medical professional can insert the tip of a marker, for example, to leave a marking on the skin of a test subject. The allergy test type indicator 808 is a barcode or other data carrier formed on the panel 802 that indicates an allergy test type identifier (e.g., 1478932). The reference maker 810 includes a stencil or apertures through which a medical professional can insert the tip of a marker, for example, to leave reference markings (e.g., crosshairs) on the skin of a test subject. The template type indicator 812 is in the form of a label adhered to the panel 802 indicating a template type identifier of “2-5-M.”

At 704, an allergy test type identifier is obtained. In the illustrated example, the processing unit 112a obtains the allergy test type identifier from the barcode reader 166. For example, an operator of the computer system 100 scans the allergy test type indicator 808 with the barcode reader 166 and a corresponding allergy test type identifier is provided to the processing unit 112a. In one embodiment, the processing unit 112a obtains the allergy test type identifier via the keyboard 146. For example, an operator of the computer system 100 enters the allergy test type identifier via the keyboard 146 in response to a prompt that is displayed by the monitor 152. In an embodiment in which the allergy test type indicator 808 is in the form of an RFID tag, the processing unit 112a may obtain the template type identifier from the RFID reader 168. For example, an operator of the computer system 100 scans the allergy test type indicator 808 with the RFID reader 168 and a corresponding allergy test type identifier is provided to the processing unit 112a. In an embodiment in which the allergy test type indicator 808 is in the form of a stencil, the processing unit 112a obtains the template type identifier from the camera unit 164. For example, after a medical professional transfers markings to the skin of a test subject using the stencils, the medical professional uses the camera unit 164 to capture an image of the markings and transfers corresponding image data to the processing unit 112a. The processing unit 112a may apply an optical character recognition algorithm to the image data, for example, to determine the allergy test type identifier.

At 706, allergy test information is obtained. In one embodiment, the processing unit 112a obtains the allergy test information from the program data 142 of the system memory 114. For example, the processing unit 112a obtains information corresponding to Table 2, which is associated with the allergy test type identifier obtained at 704. In one embodiment, the processing unit 112a obtains the allergy test information via the keyboard 146. For example, a medical professional enters the allergy test information via the keyboard 146 in response to a series of prompts that are displayed by the monitor 152.

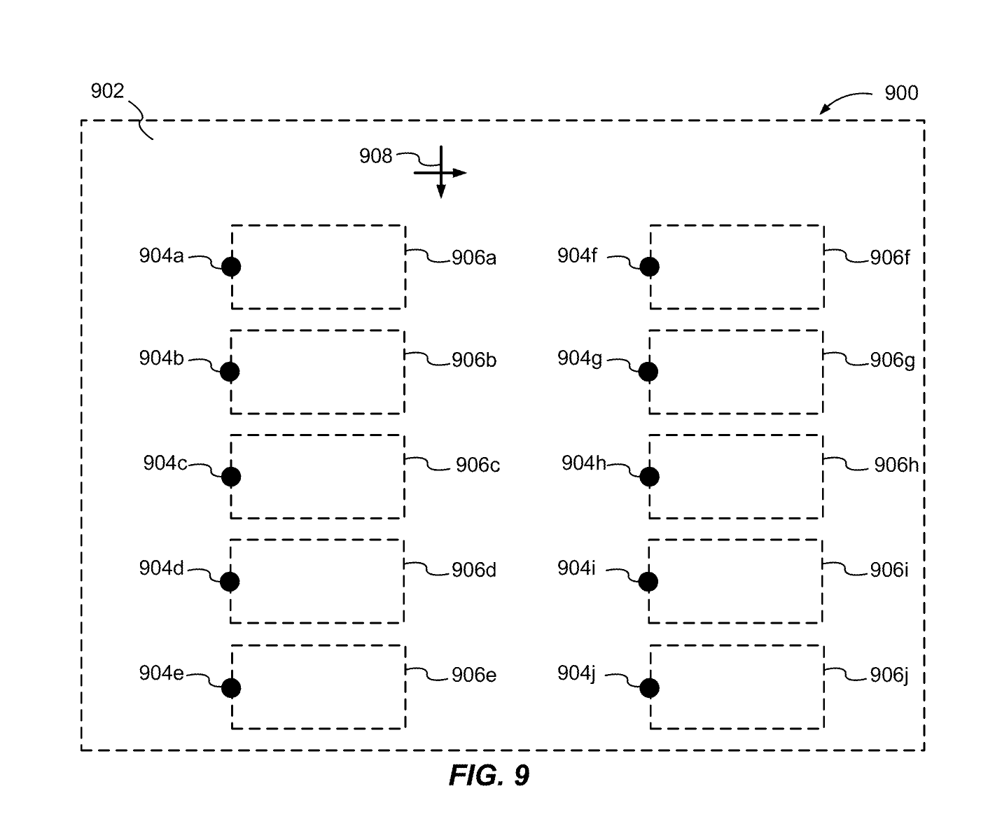

At 708, puncture site markings are formed on the skin of a test subject. In one embodiment, a medical professional places the template 800 on the skin of the test subject and inserts the tip of a marker into each of the stencils or apertures of the puncture site indicators 806a-806j to form corresponding markings on the skin of the test subject. For example, FIG. 9 shows a test area 900 on the skin 902 of the test subject. The test area 900 includes a plurality of puncture site markings 904a-904j formed using the puncture site indicators 806a-806j of the template 800. The puncture site markings 904a-904j are used to indicate a plurality of corresponding puncture site areas 906a-906j, which are not visible on the skin 902 of the test subject in the illustrated embodiment. The illustrated embodiment may also include applying a reference marking 908 to the skin of the test subject at 708. For example, a medical professional places the tip of a marker into one or more stencils or apertures of the reference marker 810 to transfer the reference marking 908 to the skin 902 of the test subject.

The method 700 may also include forming a plurality of markings corresponding to the test substance indicators 804a-804j, or portions thereof at 708. For example, markings corresponding to the index numbers of the test substance indicators 804a-804j can be formed near the puncture site markings 904a-904j to visually aid a medical professional who is applying test substances at 710, as will be described below.

The puncture site indicators 806a-806j and the reference marker 810 may include pre-inked markings on the lower surface of the panel 802 that are transferred to the skin of a test subject when a medical professional places the lower surface of the panel 802 in contact with the skin of the test subject. The puncture site indicators 806a-806j and the reference marker 810 may include projections extending from the lower surface of the panel 802 to which an ink is applied and transferred to the skin of the test subject when a medical professional places the projections in contact with the skin of the test subject to form the puncture site markings 904a-904j and the reference marking 908.

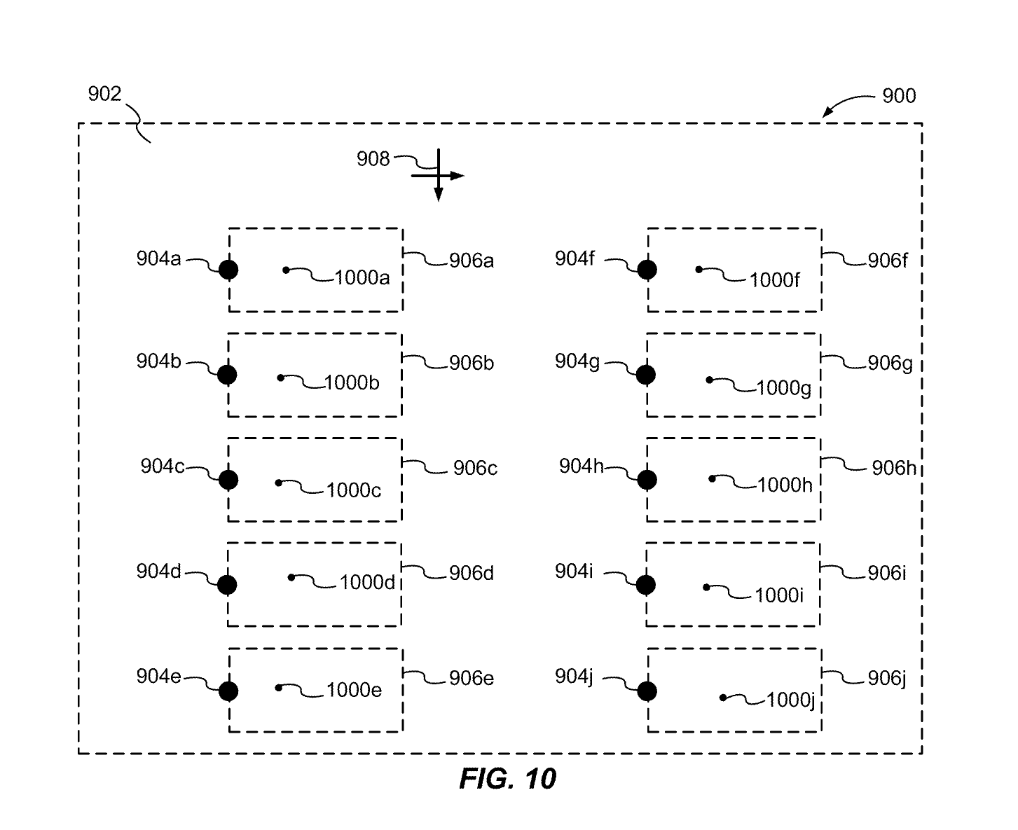

At 710, test substances are applied in the puncture site areas 906a-906j corresponding to the puncture site markings 904a-904j formed at 708. For example, FIG. 10 shows the test area 900 after a medical professional uses applicators that have been dipped into the test substances contained in the wells 406a-406j of the tray 400 to apply the test substances just beneath the skin 902 of the test subject at puncture site locations 1000a-1000j. The test substance indicators 404a-404j of the tray 400 may be the same as the test substance indicators 804a-804j of the template 800, and corresponding markings are formed near the puncture site markings 904a-904j at 708. For example, the test substance indicator 404a of the tray 400 is the number “1” and the test substance indicator 804a of the template 800 is the number “1,” which a medical professional uses to form a marking indicating the number “1” near the puncture site marking 904a on the skin 902 of the test subject. The medical professional can take note of the test substance indicator 404a (i.e., “1”) when the applicator is removed from the well 406a and then look for the same marking (i.e., “1”) in the same row as the puncture site marking 904a on the skin 902 of the test subject when applying the test substance on the tip of the applicator to help ensure that the test substance is applied to the correct area of the skin 902 of the test subject.

A predetermined amount of time (e.g., 15 minutes) after the test substances were applied at 710, image data corresponding to one or more images of the test area 900 on the skin 902 of the test subject is obtained at 712. The processing unit 112a may obtain the image data from the camera unit 164. For example, a medical professional may use the camera unit 164 to capture one or more images of the test area 900 and causes the camera unit 164 to transmit the image data to the processing unit 112a. The processing unit 112a may obtain the image data wirelessly from the camera unit 164. The processing unit 112a may obtain the image data from the memory card reader 170. For instance, a medical professional uses the camera unit 164 to capture one or more images of the test area 900 and corresponding image data is stored on a memory card that has been inserted into the camera unit 164. The memory card is subsequently removed from the camera unit 164 and inserted into the memory card reader 170, which transmits the image data to the processing unit 112a. The camera unit 164 may, in some implementations, include two cameras and the processing unit 112a obtains the image data from both cameras of the camera unit 164 at 712. The processing unit 112a may obtain the image data from the network interface 160. For example, the camera unit 164 transmits the image data to the processing unit 112 via the network interface 160.

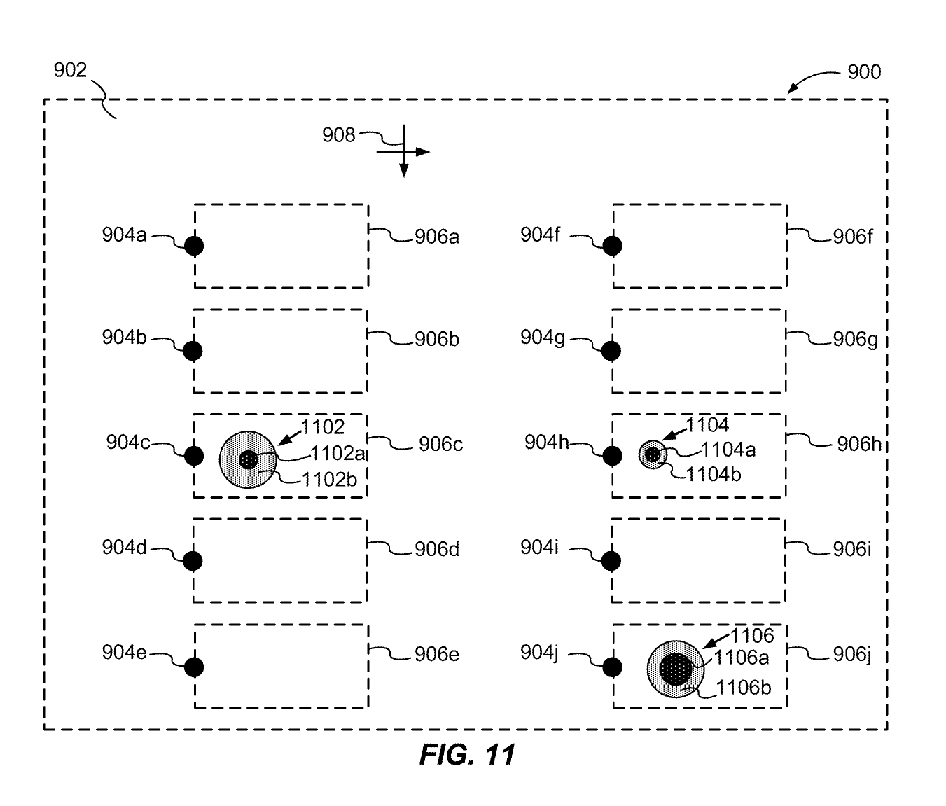

FIG. 11 illustrates the test area 900 of the skin 902 of the test subject a predetermined amount of time (e.g., 15 minutes) after the test substances were applied at 710. The test area 900 of the skin 902 of the test subject includes allergic reactions 1102, 1104, and 1106. The allergic reaction 1102 includes a wheal 1102a and a flare 1102b in the puncture site area 906c. The allergic reaction 1104 includes a wheal 1104a and a flare 1104b in the puncture site area 906h. The allergic reaction 1106 includes a wheal 1106a and a flare 1106b in the puncture site area 906j.

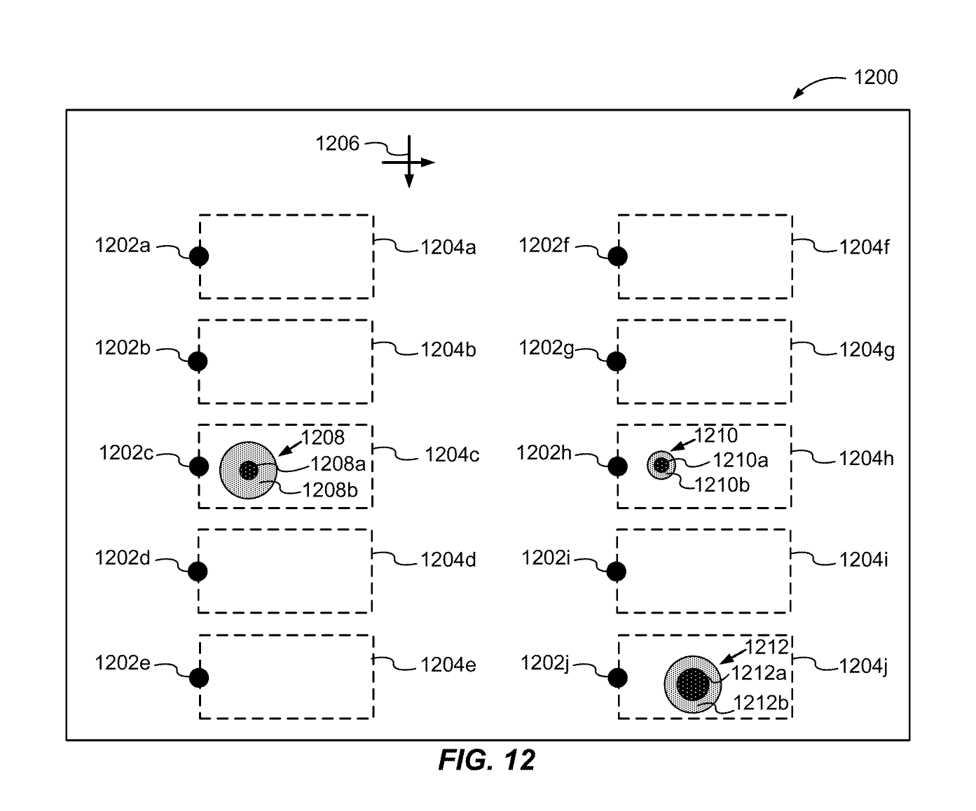

FIG. 12 illustrates an image 1200 of the test area 900 shown in FIG. 11. The image 1200 includes puncture site marking regions 1202a-1202j, corresponding puncture site area regions 1204a-1204j, a reference marking region 1206, and allergic reaction regions 1208, 1210, and 1212. The puncture site marking regions 1202a-1202j correspond to the puncture site markings 904a-904j formed on the skin 902 of the test subject. The puncture site area regions 1204a-1204j correspond to the puncture site areas 906a-906j. The reference marking region 1206 corresponds to the reference marking 908. The allergic reaction regions 1208, 1210, and 1212 correspond to the allergic reactions 1102, 1104, and 1106. The allergic reaction region 1208 includes a wheal region 1208a and a flare region 1208b in the puncture site area region 1204c. The allergic reaction region 1210 includes a wheal region 1210a and a flare region 1210b in the puncture site area region 1204h. The allergic reaction region 1212 includes a wheal region 1212a and a flare region 1212b in the puncture site area region 1204j.

At 714, the image data obtained at 710 is analyzed and a test result is determined for each of the puncture site indicators 806a-806j of the template 800 provided at 702. In one embodiment, the image data is in at least one of the following image/video formats: Joint Photographic Experts Group (JPEG), JPEG 2000, Moving Picture Experts Group (MPEG), Exchangeable image file format (Exif), Tagged Image File Format (TIFF), Graphics Interchange Format (GIF), Bit Map Picture (BMP), and Portable Network Graphics (PNG).

The processing unit 112a may execute an allergy test results application stored in the application programs 138 of the system memory 114 to analyze the image data and determine corresponding test results at 714. For example, the allergy test results application includes image processing algorithms for performing pattern recognition, object recognition, gradient matching, and/or edge matching using edge detection techniques (e.g., Canny edge detection). The processing unit 112a uses the algorithms included in the allergy test results application to identify puncture site marking regions, identify corresponding puncture site area regions, and determine whether an allergic reaction region is included in each of the puncture site area regions. For each allergic reaction region determined to be present, the processing unit 112a determines the size of a wheal region and the size of a flare region, and determines a corresponding size of a wheal and size of a flare formed on the skin 902 of the test subject, assigns a test score based on the sizes, and associates the test score with a test substance identifier.

The processing unit 112a may use image processing algorithms included in the allergy test results application to identify the puncture site marking regions 1202a-1202j included in the image data obtained at 712. The processing unit 112a then determines the boundaries of corresponding puncture site area regions 1204a-1204j according to predetermined rules of the allergy test results application. For example, the processing unit 112a uses the test information obtained at 706 (e.g., the coordinates of the puncture site indicators) to identify the puncture site marking regions 1202a-1202j and the boundaries of the corresponding puncture site area regions 1204a-1204j included in the image data obtained at 712.

The processing unit 112a may use image processing algorithms included in the allergy test results application to determine whether each of the puncture site area regions 1204a-1204j includes an allergic reaction region. For each of the puncture site area regions 1204a-1204j that the processing unit 112a determines does not include an allergic reaction region, a score of 0 is assigned to a corresponding test substance. For each of the puncture site area regions 1204a-1204j that the processing unit 112a determines includes an allergic reaction region, the processing unit 112a determines a size (e.g., length, width, height, and/or diameter) of a wheal region and a size (e.g., length, width, height, and/or diameter) of a flare region. As previously noted, the camera unit 164 may include two or more cameras and the processing unit 112a generates three-dimensional data from the image data received at 712. The processing unit 112a uses the three-dimensional data to determine a length, width, height, and/or a diameter of each wheal and flare, which the processing unit 112a uses to determine the test results at 714.

The sizes of each wheal region and flare region are then scaled to determine the actual sizes of a corresponding wheal and flair on the skin 902 of the test subject. For example, the processing unit 112a performs scaling using a scaling factor that is determined by comparing a size of the reference marker region 1206 to a predetermined size (e.g., length, width, height, and/or diameter) of the reference marker 810 of the template 800. The information may, for instance, represent the predetermined size, shape, and color of the reference marker 810 is included in the layout information associated with the template 800. In some implementations, the processing unit 112a determines a scaling factor by comparing one or more sizes of one or more of the puncture site marking regions 1202a-1202j to one or more sizes of one of more of the test substance indicators 804a-804j of the template 800.

After the actual sizes of each wheal and flare are determined, the processing unit 112a determines a test result or score using an allergy test result algorithm of the allergy test results application. The processing unit 112a may employ a lookup table stored in the program data 142 of the system memory 114 to determine a test result for each allergic reaction. For example, the lookup table indicates that an allergic reaction having a wheal size of 4 millimeters and a flare size of 8 millimeters is assigned a score of 3. In one embodiment, the program data 142 of the system memory 114 stores a plurality of lookup tables, each of which is associated with one or more of the test substance identifiers. The processing unit 112a may use a lookup table associated with a particular substance identifier to determine a test result corresponding to the substance identifier. For example, the processing unit 112a uses the allergy test information obtained at 706 to determine that a particular puncture site area region corresponds to a bean test substance identifier, and uses a lookup table associated therewith to determine the test result for the bean test substance. In one embodiment, the processing unit 112a assigns a test result, at least in part, by comparing the determined sizes of the wheal and flare for a particular allergic reaction to the sizes of the wheal and flare determined for a positive and/or negative control substance.

After the test results are determined for each of the puncture site area regions 1204a-1204j, the processing unit 112a uses the test information obtained at 706 to determine a substance identifier corresponding to each of the puncture site area regions 1204a-1204j. Each of the test substance identifiers is then associated with the test result that was determined for the corresponding one of the puncture site area regions 1204a-1204j. The test results may be stored in the program data 142 of the system memory 114 (and/or another computer-readable storage medium), displayed by the monitor 152, printed by a printer, and/or transmitted via the network interface 160.

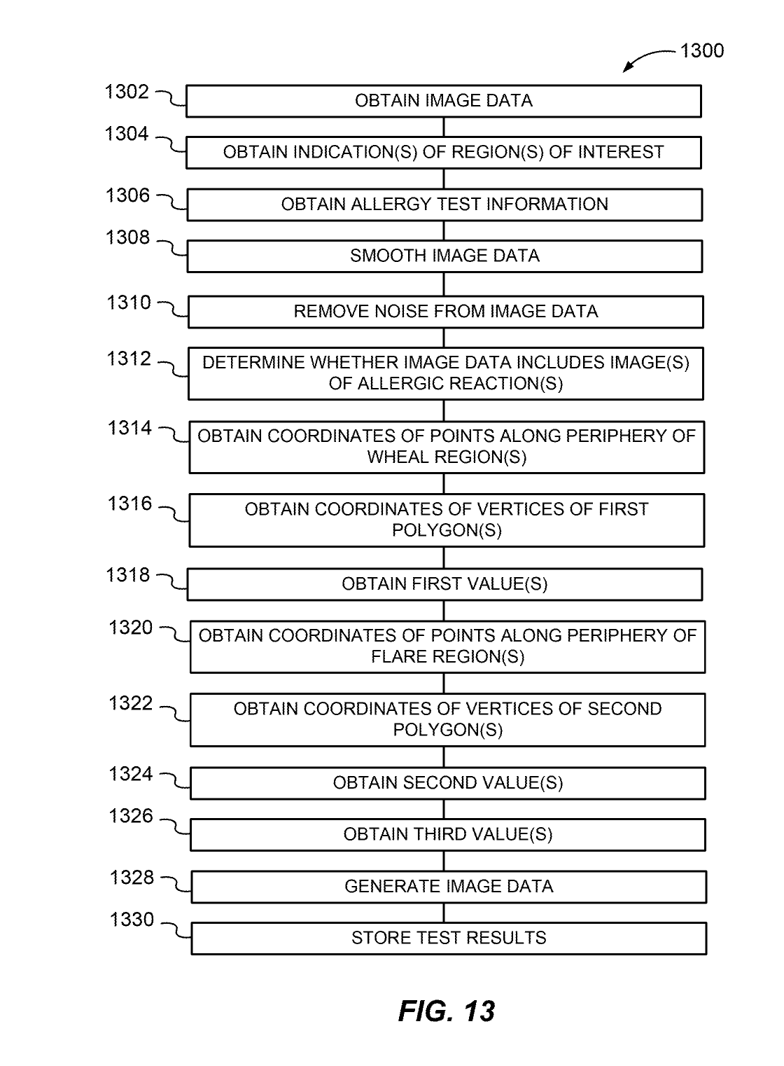

A method of generating test results according to one illustrated embodiment will now be described with reference to FIGS. 13-20. FIG. 13 shows a method 1300 that may be performed by a computer system (e.g., computer system 100) to generate test results. At 1302, the computer system obtains image data. For example, the processing unit 112a may obtain the image data via the system bus 116 from one or more of the RAM 120, the hard disk drive 124, the optical disk drive 128, the network interface 160, the camera unit 164, and the memory card reader 170.

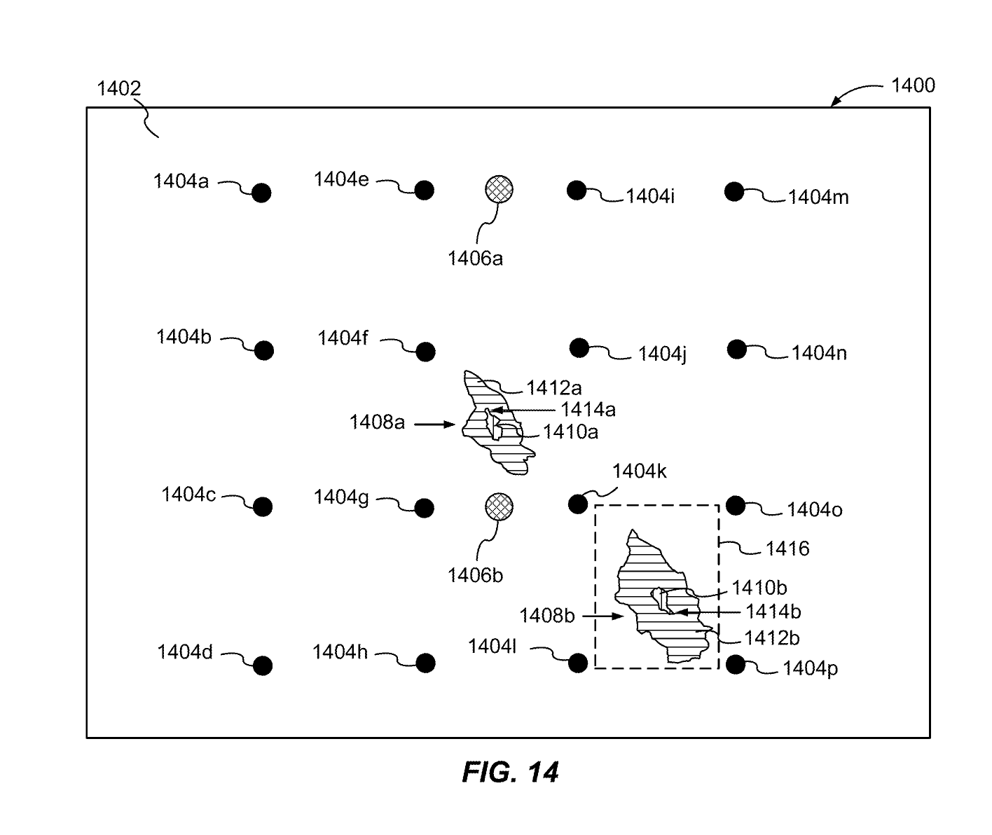

FIG. 14 is a diagram of a test area 1400 of the skin 1402 of a test subject after several allergens have been introduced to the skin 1402, according to one illustrated embodiment. The test area 1400 includes test area markers 1404a-1404p and fiducial markers 1406a and 1406b on the skin 1402 of the test subject. The fiducial markers 1406a and 1406b have predetermined colors, shapes, and sizes; for example, each of the fiducial markers 1406a and 1406b may be a green circle having a diameter of a predetermined length. The test area 1400 also includes allergic reactions 1408a and 1408b on the skin 1402 of the test subject. The allergic reaction 1408a includes a wheal region 1410a and a flare region 1412a, and the allergic reaction 1408b includes a wheal region 1410b and a flare region 1412b. The wheal region 1410a includes a pseudopod region 1414a, and the wheal region 1410b includes a pseudopod region 1414b, wherein a pseudopod is a slender extension from the edge of a wheal. The image data obtained at 1302 may correspond to an image of the test area 1400, for example.

At 1304, the computer system obtains one or more indications of one or more regions of interest. For example, the processing unit 112a may instruct the video interface 154 to cause the first image data to be displayed by the monitor 152, and an operator may depress a button on the mouse 148 and move the mouse to 148 to cause an indication of a region of interest 1416 to be displayed around the image of the allergic reaction 1408b displayed by the monitor 152. For example, the operator may cause a bounding box indicated by the dashed lines in FIG. 14 to be displayed on the monitor 152. In response, the processing unit 112a may obtain coordinates corresponding to the upper left and lower right corners of the bounding box, which indicates the region of interest 1416. For example, the processing unit 112a may determine the boundaries of the region of interest 1416 by determining the boundaries of a rectangle having upper left and lower right corners at the obtained coordinates.

In the illustrated example, only one region of interest 1416 is described; however, the operator could indicate a number of regions of interest, for example, by performing a number of the above-described operations using the mouse 148 or other pointing device or mechanism, including gestures. Alternatively, the operator may indicate that a region of interest corresponds to the entire test area 1400, for example, by selecting a menu item that is displayed by the monitor 152 using the mouse 148.

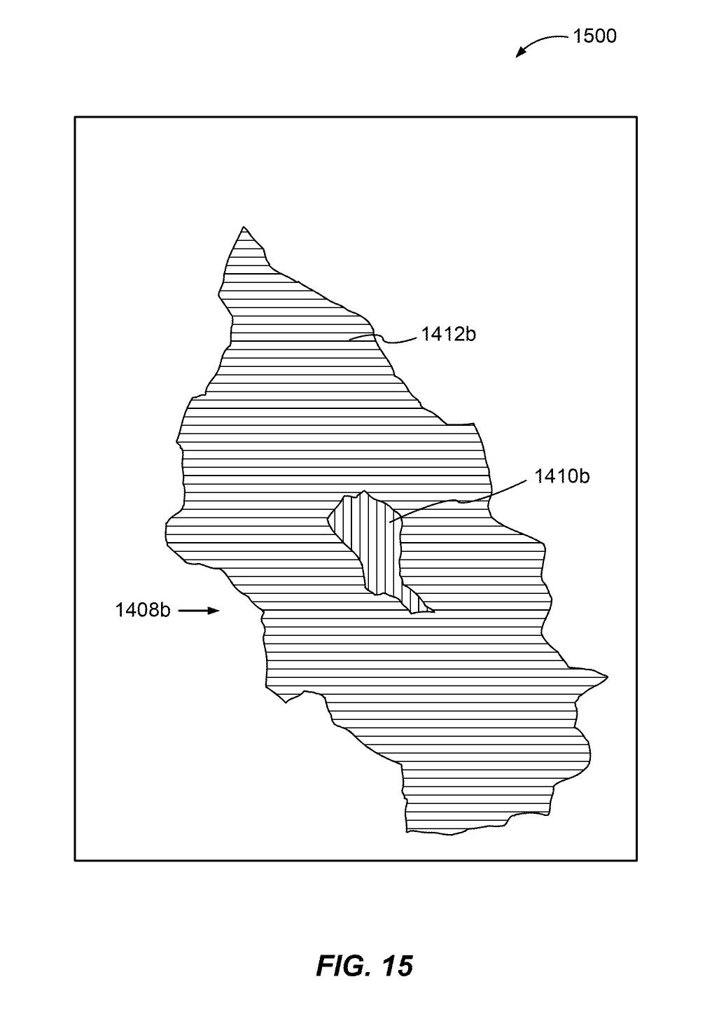

In one implementation, after the computer system obtains each indication of a region of interest, the processing unit generates image data corresponding to each region of interest. For example, after the processing unit 112a obtains the indication of the region of interest 1416, the processing unit 112a generates image data that corresponds to a portion of the test area 1400 cropped based on the region of interest 1416. FIG. 15 shows a diagram 1500 of a portion of the test area 1400 cropped based on the region of interest 1416. That is, the processing unit 112a may generate image data that corresponds to the diagram 1500.

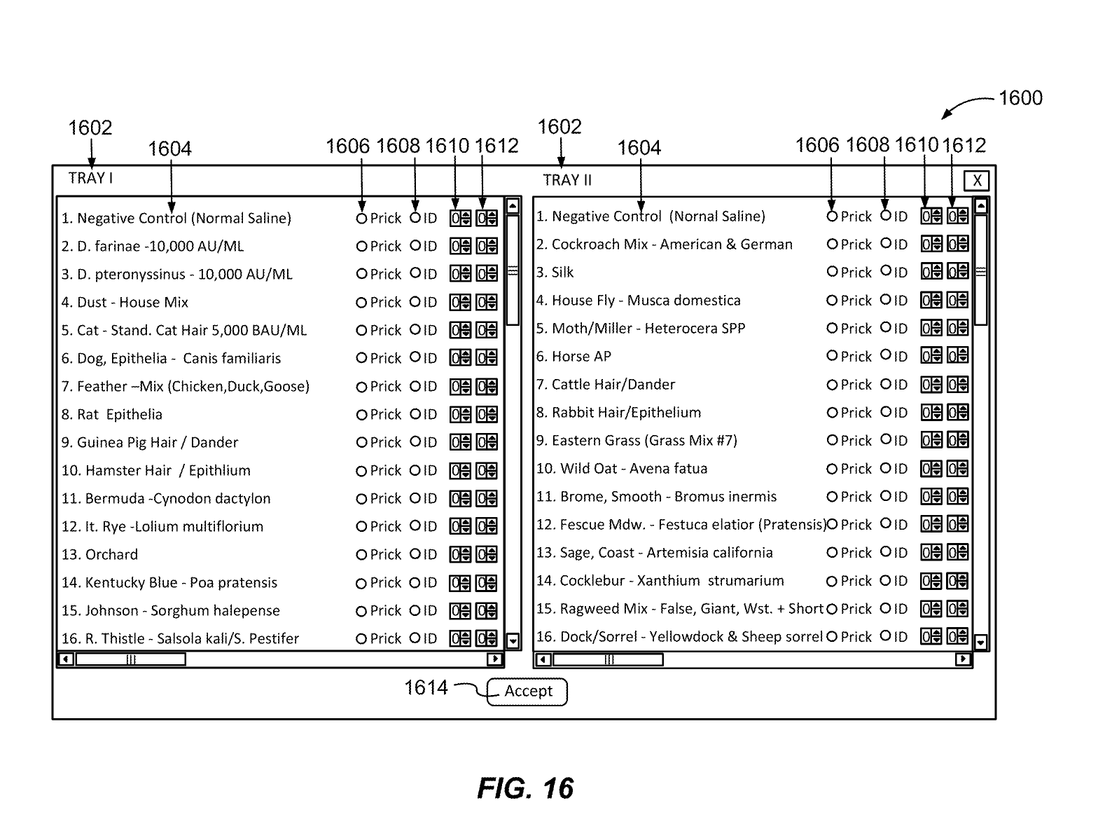

At 1306, the computer system obtains allergy test information. For example, the processing unit 112a may obtain allergy test information in response to an operator making a number of selections via an operator interface 1600 shown in FIG. 16, which may be displayed by the monitor 152. The operator interface 1600 includes a plurality of allergen tray identifiers 1602, a plurality of allergen identifiers 1604 (only two called out in FIG. 16), a plurality of user selectable radio buttons 1606 (only two called out in FIG. 16), a plurality of user selectable radio buttons 1608 (only two called out in FIG. 16), a plurality of user fillable text boxes 1610 (only two called out in FIG. 16), a plurality of user fillable text boxes 1612 (only two called out in FIG. 16), and a user selectable button 1614. The user may select radio buttons 1606 to indicate a puncture or a prick type of allergic reaction test and select the radio buttons 1608 to indicate an intradermal type of allergic reaction test.

For example, if the allergic reaction 1408b resulted from puncturing or pricking the skin 1402 of the test subject with an applicator and introducing an allergen comprising D. farinae, an operator may select the radio button 1606 in the same row as the “D. farinae-10,000 AU/ML” allergen identifier 1604. Additionally, the operator may enter “1” into the text box 1610 and enter “3” into the text box 1612 in the same row as the “D. farinae-10,000 AU/ML” allergen identifier 1604 to specify coordinates (e.g., a row and a column) corresponding to the region of interest 1416. That is, the region of interest 1416 is in the first row and the third column defined by the test area markers 1404a-1404p. In response to the operator selecting the “Accept” button 1614, the processing unit 112a obtains test information including identifiers of the radio button(s) 1606 and/or 1608 that have been selected and the text boxes 1610 and 1612 into which text has been entered, and the corresponding text that has been entered into the text boxes 1610 and 1612. The processing unit 112a may cause the test information to be stored in RAM 120, for example.

Additionally, the computer system may obtain color information at 1306, wherein the color information indicates colors for displaying first and second polygons, which will be explained below. For example, the processing unit 112a may display an operator interface (not shown) that enables an operator to specify (e.g., select from a list) a first color for displaying the first polygons and a second color for displaying the second polygons. After the operator has selected the colors, the processing unit 112a may receive indications (e.g., textual descriptions or numeric identifiers) of those colors.

At 1308, the computer system smooths the image data corresponding to at least one region of interest. For example, the processing unit 112a may smooth the image data corresponding to the region of interest 1416 by processing the image data using a conventional “rectangular” or “unweighted sliding-average smooth” image processing algorithm.

At 1310, the computer system removes noise from the image data corresponding to at least one region of interest. For example, the processing unit 112a may remove noise from the image data corresponding to the region of interest 1416 by processing the image data using a conventional algorithm for convolving the image data with a mask that represents a low-pass filter.

At 1312, the computer system determines whether the image data corresponding to at least one region of interest includes one or more images of one or more allergic reactions. For example, the processing unit 112a may process pixel values included in the image data corresponding to the region of interest 1416 to determine whether discoloration of the skin 1402 has occurred that is consistent with an allergic reaction, as defined by predefined criteria such as a minimum amount of discoloration and a minimum area of discoloration.

At 1314, the computer system obtains coordinates of points along the periphery of one or more images of one or more wheal regions included in the image data. In one implementation, the image data is stored in a three-dimensional array of pixel values having indices x, y, and z, wherein the values of the indices x and y corresponds to a position in a rectangular region, and the value of the index z corresponds to a particular color (e.g., red, green, or blue). The coordinates of a particular pixel or point in an image depends on the values of the indices x and y for that pixel or point. For example, a pixel or point having coordinates (0,0) (i.e., indices of x=0 and y=0) may be located in the upper left corner of a rectangular region corresponding to the image.

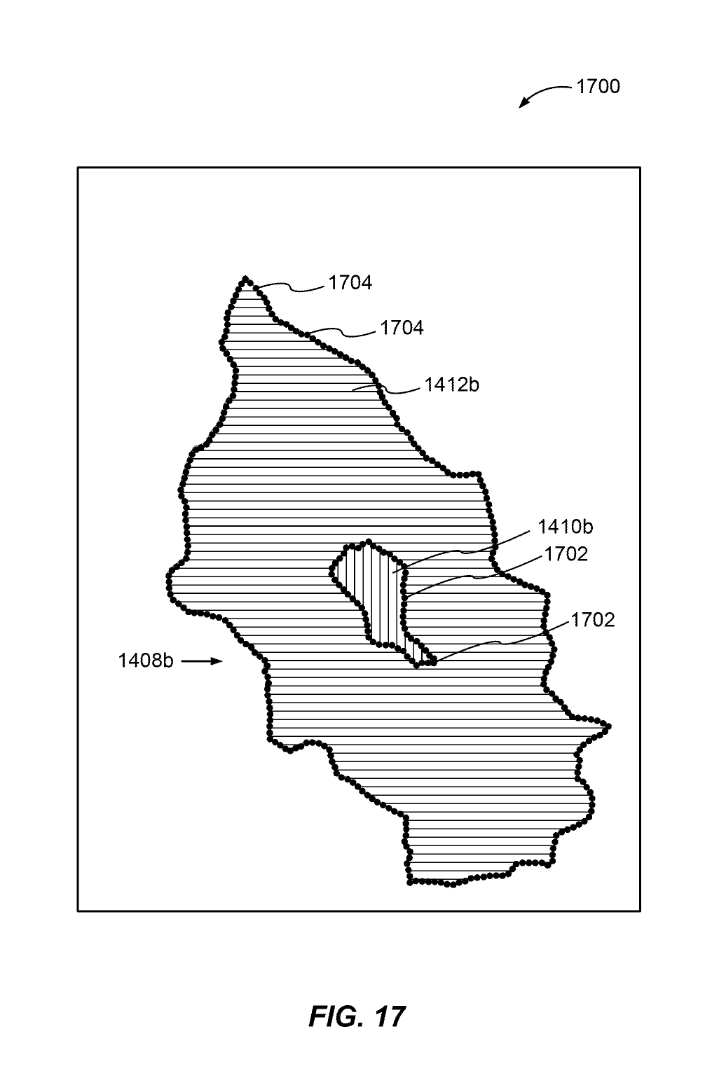

For example, at 1314, the processing unit 112a may process the pixel values included in the image data corresponding to the region of interest 1416 to identify pixels where the corresponding colors are indicative of a transition from a color associated with a flare to a color associated with a wheal, and store an array of coordinates corresponding to those pixels, for example, in RAM 120. FIG. 17 shows a diagram 1700 that includes the allergic reaction 1408b with a plurality of points 1702 (only two called out in FIG. 17) disposed about the periphery of the wheal region 1410b. The processing unit 112a may obtain the coordinates of the points 1702 at 1314, for example. Additionally, the processing unit 112a may determine the distance between points in the image, for example, based on a number of pixels comprising the diameter of the fiducial marker 1406b, which has a predetermined distance or length. Accordingly, the processing unit 112a may determine the distance between points in the image based on the coordinates of those points.

At 1316, the computer system obtains coordinates of vertices of one or more first polygons. For example, the processing unit 112a performs an Ant Colony Optimization (ACO) using the coordinates of the points 1702 obtained at 1314. An ACO is an Artificial Intelligence technique that can be employed to construct the first polygon. The biological idea behind the ACO meta-heuristic is to simulate the behavior of a colony of individual ants when they are looking for food. Real ants in nature search for food in a random proximity to their nests. Once an ant finds a source of food, it evaluates a quality and a quantity of the source of food. Then, in a path back to the nest, each ant deposits a chemical pheromone trail on the ground, in order to guide other ants in the colony to the source of food.

Inspired in this behavior, the ACO algorithm is based on a fully constructive model in which each of a plurality of artificial ants builds a solution to the problem by exploring a construction graph. Each artificial ant moves from one state to another during a search process, wherein states denote points bounding desired regions (e.g., a wheal region or a flare region). In general terms, a preference for moving from one node to another depends on values associated with each pathway included in artificial information and heuristic information. The artificial information is based in the pheromone trails deposited by the artificial ants and is iteratively updated by ants during the search process. The heuristic information is related to an application domain denoting a preference for moving from one state to another. It is noted that, because the heuristic information is known in advance (i.e., a distance in pixels between two points), the heuristic information may not be updated during the search process.

After the construction phase is complete, pheromone trails are updated using solutions found by ants. A pheromone evaporation phase is implemented in which the pheromone trails are uniformly reduced. Additionally, one or more solutions found are used to increase the values of paths included in selected solutions. At the end of the algorithm, the optimal sequence of points bounding a desired region is returned. That is, after all points 1702 bounding the wheal region 1410b are obtained at 1314, a subset of those points 1702 in a particular sequence is obtained as the vertices of the first polygon at 1316. Selecting an optimal sequence of the points 1702 requires solving a discrete combinatorial problem. To solve this problem, software stored in the applications programs 138, for example, implements a variant of Ant Colony Optimization (ACO) called a MAX-MIN Ant System which is capable of efficiently solving the optimization task.

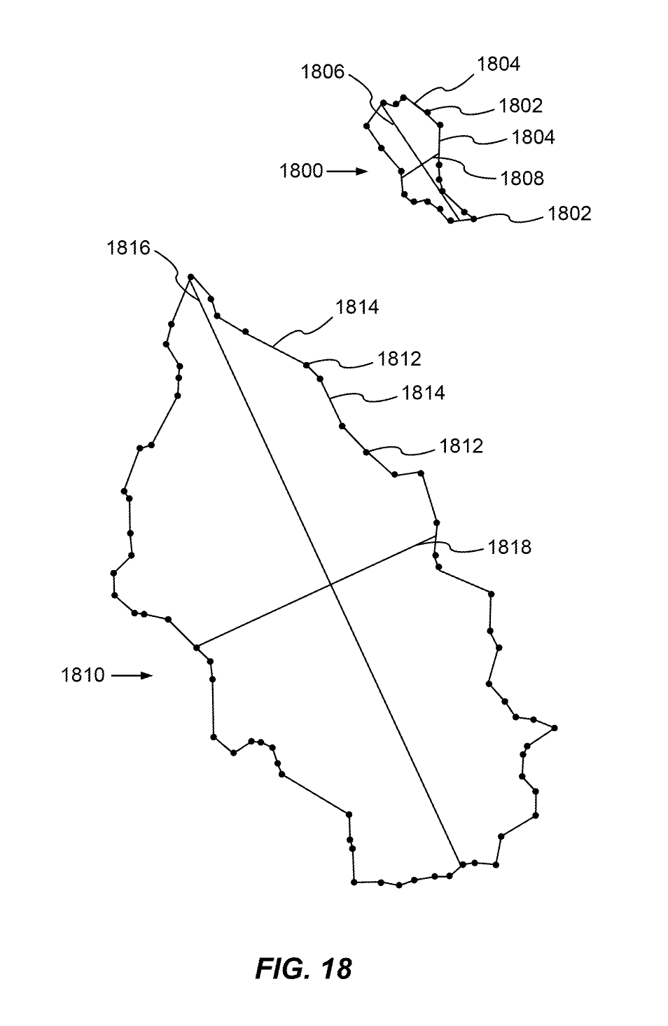

For example, after the processing unit 112a performs a first Ant Colony Optimization using the coordinates of the points 1702 obtained at 1314, coordinates of a plurality of vertices 1802 are obtained at 1316. FIG. 18 shows an example of the vertices 1802 (only two called out in FIG. 18). The vertices 1802 are arranged in an order such that, when sequential pairs of the vertices 1802 are interconnected by a plurality of line segments 1804, a first polygon 1800 is formed. That is, the first polygon 1800 is formed when a first vertex 1802 in a sequence and a second vertex in the sequence 1802 are interconnected by a first line segment 1804, the second vertex 1802 in the sequence and a third vertex 1802 in the sequence are interconnected by a second line segment 1804, etc.

At 1318, the computer system obtains one or more first values. The computer system may obtain the one or more first values by processing the coordinates of the vertices of the one or more first polygons obtained at 1316 using an algorithm implemented in software stored by the computer system. The algorithm may approximate both wheal and flare regions, and also calculate their real size (e.g., in millimeters). For example, the processing unit 112a uses the coordinates of vertices 1802 obtained at 1316 to determine a first line segment 1806 that corresponds to a first dimension of the first polygon 1800 and a second line segment 1808 that corresponds to a second dimension of the first polygon 1800, as shown in FIG. 18. In one implementation, the first line segment 1806 corresponds to a longest dimension of the first polygon 1800 and the second line segment 1808 is orthogonal to the first line segment 1806 and passes through a midpoint (e.g., center) of the first line segment 1806. The processing unit 112a then obtains a first value, for example, by determining the length of the first line segment 1806 and the length of the second line segment 1808 and summing those lengths.

At 1320, the computer system obtains coordinates of points along the periphery of one or more flare regions. For example, the processing unit 112a may process the values of the pixels included in the image data corresponding to the region of interest 1416 to identify pixels where the corresponding colors are indicative of a transition from a color associated with the skin 1402 to a color associated with a flare, and store an array of coordinates corresponding to those pixels, for example, in RAM 120. FIG. 17 shows the allergic reaction 1408b with a plurality of points 1704 (only two called out in FIG. 17) disposed about the periphery of the wheal region 1412b. The processing unit 112a may obtain the coordinates of the points 1704 at 1320, for example.

At 1322, the computer system obtains coordinates of vertices of one or more second polygons. For example, the processing unit 112a performs a second Ant Colony Optimization using the coordinates of the points 1704 obtained at 1320 to obtain the coordinates of a plurality of vertices 1812, as shown in FIG. 18, in a manner similar to that described above in connection with 1316.

At 1324, the computer system obtains one or more second values. For example, the processing unit 112a uses the coordinates of the vertices 1812 obtained at 1322 to determine a first line segment 1816 that corresponds to a first dimension of the second polygon 1810 and a second line segment 1818 that corresponds to a second dimension of the second polygon 1810, as shown in FIG. 18. In one implementation, the first line segment 1816 corresponds to a longest dimension of the second polygon 1810 and the second line segment 1818 is orthogonal to the first line segment 1816 and passes through a midpoint (e.g., center) of the first line segment 1816. The processing unit 112a then obtains a second value, for example, by determining the length of the first line segment 1816 and the length of the second line segment 1818 and summing those lengths.

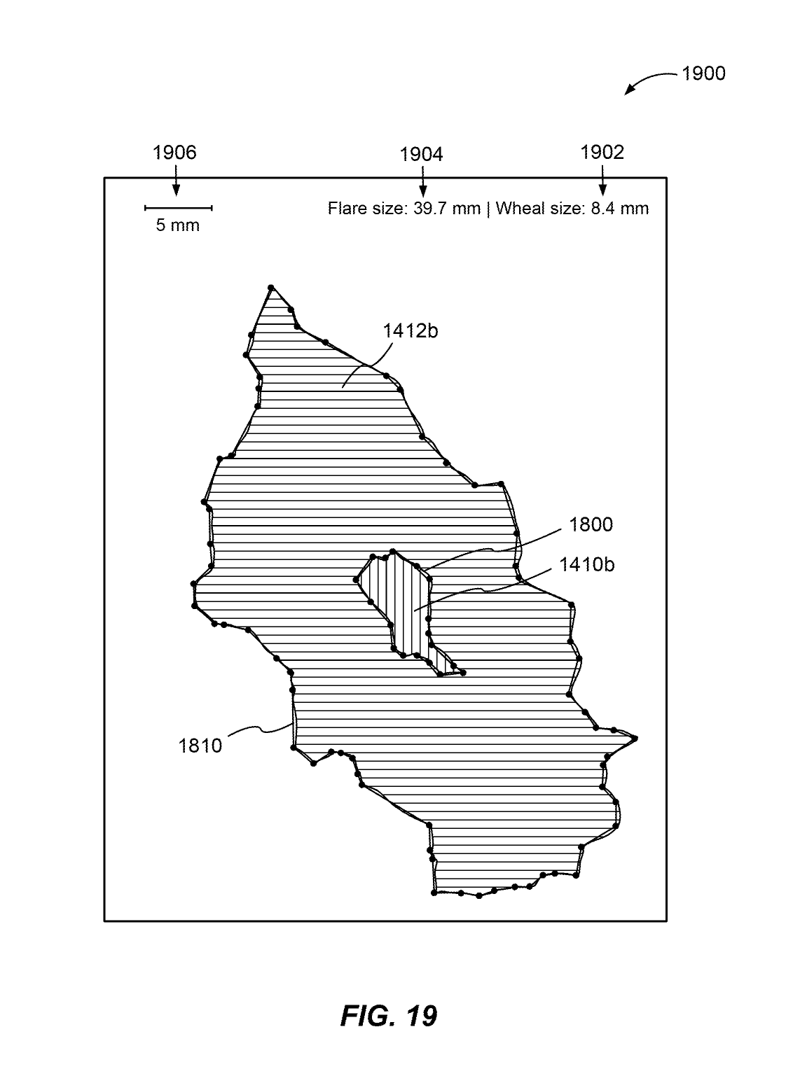

At 1328, the computer system generates image data that includes one or more first polygons and one or more second polygons. For example, the processing unit 112a generates image data corresponding to a test result 1900 shown in FIG. 19. The test result 1900 includes the first polygon 1800 mapped onto the wheel region 1410b and the second polygon 1810 mapped onto the flare region 1412b. The test result 1900 also may include a first value 1902 (e.g., 8.4), a second value 1904 (e.g., 39.7), and a marker 1906 that indicates the scale of the test result 1900.

The first polygon 1800 and the second polygon 1810 may have the first color and the second color, respectively, indicated in the color information obtained at 1306. For example, the processing unit 112a may generate the image data at 1328 such that the image data includes an indication that the first polygon 1800 has the first color indicated in the test information and an indication that the second polygon 1810 has the second color indicated in the test information.

At 1330, the computer system stores test results. For example, the processing unit 112a causes the image data corresponding to the test result 1900 generated at 1328 to be stored by the hard disk drive 124. Additionally, the processing unit 112a may cause at least some of the test information obtained at 1306, the first value(s) obtained at 1318, and the second value(s) obtained at 1324 to be stored by the hard disk drive 124.

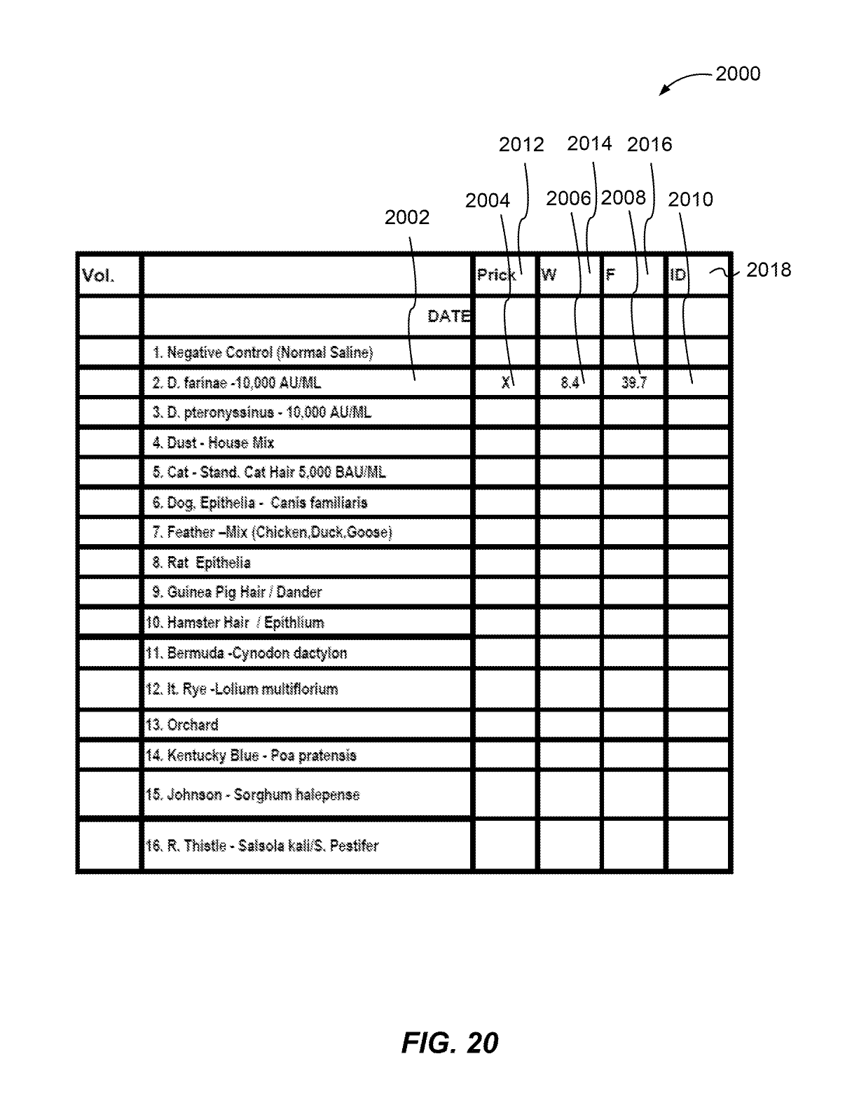

For example, the processing unit 112a may store test results in a spreadsheet 2000 that includes a plurality of cells, as shown in FIG. 20. If an operator had selected the radio button 1606 in the same row as the “D. farinae-10,000 AU/ML” allergen identifier 1604 in the operator interface 1600, the processing unit 112a may cause a predetermined character or value (e.g., “X”) to be stored in a cell 2004, which is in the same row as a cell 2002 that includes the text “D. farinae-10,000 AU/ML” and in the same column as a cell 2012 that includes the text “Prick”, which may be used to indicate a puncture or prick type of allergic reaction test. That is, a value stored in the cell 2004 may indicate that the allergen “D. farinae-10,000 AU/ML” was introduced to the skin of a test subject via a puncture or a prick type of allergic reaction test. If an operator had selected the radio button 1608 in the same row as the “D. farinae-10,000 AU/ML” allergen identifier 1604 in the operator interface 1600, the processing unit 112a may cause a predetermined character or value (e.g., “X”) to be stored in a cell 2010, which is in the same row as the cell 2002 that includes the text “D. farinae-10,000 AU/ML” and in the same column as a cell 2018 that includes the text “ID”, which may be used to indicate an intradermal type of allergic reaction test. That is, a value stored in the cell 2010 may indicate that the allergen “D. farinae-10,000 AU/ML” was introduced to the skin of a test subject via an intradermal type of allergic reaction test.

Additionally, the processing unit 112a may cause a first value (e.g., 8.4) obtained at 1318 to be stored in a cell 2006, which is in the same row as the cell 2002 that includes the text “D. farinae-10,000 AU/ML” and in the same column as a cell 2014 that includes the text “W”, which may be used to indicate a wheal dimension. Additionally, the processing unit 112a may cause a second value (e.g., 39.7) obtained at 1324 to be stored in a cell 2008, which is in the same row as the cell 2002 that includes the text “D. farinae-10,000 AU/ML” and in the same column as a cell 2016 that includes the text “F”, which may be used to indicate a flare dimension.

At 1330, the computer system also may obtain and store one or more third values based at least in part on the one or more first values obtained at 1318 and the one or more second values obtained at 1324. For example, if a first value is less than or equal to a first predetermined value (e.g., 2 mm) and a second value is less than or equal to a second predetermined value (e.g., 21 mm), the processing unit 112a may obtain a third value having a value of “1”. If the first value is less than or equal to a third predetermined value (e.g., 3 mm) and the second value is greater than the second predetermined value, the processing unit 112a may obtain a third value having a value of “2”. If the first value is greater than the third predetermined value and the second value is greater than the second predetermined value and less than or equal to a fifth predetermined value (e.g., 8 mm), the processing unit 112a may obtain a third value having a value of “3”. If the first value is greater than the third predetermined value and the second value is greater than the fifth predetermined value and a corresponding wheal region is determined not to include a pseudopod, the processing unit 112a may obtain a third value having a value of “4”. If the first value is greater than the third predetermined value and the second value is greater than the fifth predetermined value and a corresponding wheal region is determined to include at least one pseudopod, the processing unit 112a may obtain a third value having a value of “5”. Of course, the processing unit 112a may use different predetermined values and obtain different values for the third value without departing from the scope of the present application.

Various components described herein may advantageously be provided as a kit. The kit may, for example, include a template having puncture site indicators, a CDROM storing allergy test information, and instructions. The allergy test information may associate the puncture site indicators of the template with predetermined locations on the template and/or identifiers of test substance that are to be used in a particular type of allergy skin test. The CDROM may also store one or more computer programs that can cause a computer (e.g., computer system 100) to generate additional allergy test information and/or determine results of allergy skin tests performed using the template. The kit may also include a tray having wells corresponding to the puncture site markers of the template for holding test substances. Labels including names or other indicators of the test substances may be provided near corresponding wells. The kit may also include a fiducial marker that can be placed on the skin of a test subject in the field of view of an imaging system (e.g., camera unit 164) before image data corresponding to one or more images of the skin of the test subject are obtained. Image data corresponding to the fiducial marker may be used to determine image regions corresponding to puncture sites and/or to ascertain dimensions of allergic reactions that are used to determine allergy test results.

The various embodiments described above can be combined to provide further embodiments. All of the commonly assigned US patent application publications, US patent applications, foreign patents, foreign patent applications and non-patent publications referred to in this specification and/or listed in the Application Data Sheet, including but not limited to U.S. Provisional Application Ser. No. 61/857,108, filed Jul. 22, 2013 and U.S. Provisional Application Ser. No. 62/002,094, filed May 22, 2014 are incorporated herein by reference, in their entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An allergy testing system, comprising:
at least one processor;
at least one non-transitory processor-readable storage medium that stores processor-executable instructions that, when executed, cause the at least one processor to:
obtain first image data that corresponds to an image which includes a wheal region and a flare region;
obtain a first plurality of coordinates that respectively correspond to a first plurality of points disposed about a periphery of the wheal region, the first plurality of coordinates which define a plurality of vertices of a first polygon;
obtain a second plurality of coordinates that respectively correspond to a second plurality of points disposed about a periphery of the flare region, the second plurality of coordinates which define a plurality of vertices of a second polygon;
obtain a first value based at least in part on the first plurality of coordinates; and
obtain a second value based at least in part on the second plurality of coordinates.

2. The allergy testing system of claim 1 wherein the at least one storage medium stores instructions that, when executed, further cause the at least one processor to:
cause at least the first value and the second value to be stored in one or more processor-readable storage media.

3. The allergy testing system of claim 1 wherein the at least one storage medium stores instructions that, when executed, further cause the at least one processor to:
obtain a third plurality of coordinates that respectively correspond to a third plurality of points disposed about the periphery of the wheal region, wherein to obtain the first plurality of coordinates the at least one processor performs a first ant colony optimization using at least some of the third plurality of coordinates; and
obtain a fourth plurality of coordinates that respectively correspond to a fourth plurality of points disposed about the periphery of the flare region, wherein to obtain the second plurality of coordinates the at least one processor performs a second ant colony optimization using at least some of the fourth plurality of coordinates.

4. The allergy testing system of claim 1 wherein to obtain the first value the at least one processor at least in part determines a first plurality of dimensions of the first polygon, and to obtain the second value the at least one processor at least in part determines a second plurality of dimensions of the second polygon.

5. The allergy testing system of claim 4 wherein to obtain the first value the at least one processor at least in part determines a sum of the first plurality of dimensions of the first polygon, and to obtain the second value the at least one processor at least in part determines a sum of the second plurality of dimensions of the second polygon.

6. The allergy testing system of claim 1 wherein to obtain the first value the at least one processor at least in part determines a length of a first line segment that corresponds to a first dimension of the first polygon and a length of a second line segment that corresponds to a second dimension of the first polygon, the second line segment orthogonal to the first line segment, and to obtain the second value the at least one processor at least in part determines a length of a third line segment that corresponds to a first dimension of the second polygon and a length of a fourth line segment that corresponds to a second dimension of the second polygon, the fourth line segment orthogonal to the third line segment.

7. The allergy testing system of claim 6 wherein the first dimension of the first polygon is a longest dimension of the first polygon and the second line segment passes through a midpoint of the first line segment, and the first dimension of the second polygon is a longest dimension of the second polygon and the fourth line segment passes through a midpoint of the third line segment.

8. The allergy testing system of claim 1 wherein the first plurality of coordinates is arranged in a first sequence such that when sequential pairs of the first plurality of coordinates are interconnected with a first plurality of line segments the first polygon is formed, and the second plurality of coordinates is arranged in a second sequence such that when sequential pairs of the second plurality of coordinates are interconnected with a second plurality of line segments the second polygon is formed.

9. The allergy testing system of claim 1 wherein the at least one storage medium stores instructions that, when executed, further cause the at least one processor to:
generate second image data that corresponds to an image that includes the wheal region, the flare region, the first polygon, and the second polygon.

10. The allergy testing system of claim 9 wherein the at least one storage medium stores instructions that, when executed, further cause the at least one processor to:
cause the second image data to be displayed.

11. The allergy testing system of claim 10 wherein the at least one storage medium stores instructions that, when executed, further cause the at least one processor to:
obtain an indication of a first color; and
obtain an indication of a second color, wherein the second image data includes an indication that the first polygon has the first color and an indication that the second polygon has the second color.

12. The allergy testing system of claim 1 wherein the at least one storage medium stores instructions that, when executed, further cause the at least one processor to:
generate second image data that corresponds to an image that includes the wheal region, the flare region, the first polygon, the second polygon, the first value, and the second value.

13. The allergy testing system of claim 1 wherein the at least one storage medium stores instructions that, when executed, further cause the at least one processor to:
obtain an indication of a region of interest, wherein first plurality of coordinates and the second plurality of coordinates are obtained based on at least part of the first image data which corresponds to at least part of the image within the region of interest.

14. The allergy testing system of claim 1 wherein the at least one storage medium stores instructions that, when executed, further cause the at least one processor to:
receive test information; and
cause the first value, the second value, and at least some of the test information to be stored in one or more processor-readable storage media.

15. The allergy testing system of claim 14 wherein the test information includes at least one of: an indication of a type of allergen, an indication of a puncture or a prick type of allergic reaction test, an indication of an intradermal type of allergic reaction test, and an indication of coordinates that correspond to a region of an image.

16. The allergy testing system of claim 1 wherein the at least one storage medium stores instructions that, when executed, further cause the at least one processor to:
perform at least one operation on the first image data that smooths the first image data.

17. The allergy testing system of claim 1 wherein the at least one storage medium stores instructions that, when executed, further cause the at least one processor to:
perform at least one operation on the first image data that reduces noise in the image that corresponds to the first image data.

18. The allergy testing system of claim 1 wherein the at least one storage medium stores instructions that, when executed, further cause the at least one processor to:
obtain a third value based at least in part on the first value and the second value.
